(19) United States
(12) Patent Application Publication
(10) Pub. No.: US 2017/0184543 A1
(43) Pub. Date: Jun. 29, 2017

(54) ISOTACHOPHORETIC DEVICE AND METHODS

(71) Applicant: UNIVERSITY OF WASHINGTON, Seattle, WA (US)

(72) Inventors: Jonathan POSNER, Seattle, WA (US); Babak MOGHADAM, Seattle, WA (US); Kelly CONNELLY, Los Angeles, CA (US)

(21) Appl. No.: 15/300,390

(22) PCT Filed: Apr. 15, 2015
(86) PCT No.: PCT/US2015/026035

§ 371 Date: Sep. 29, 2016

Related U.S. Application Data
(60) Provisional application No. 61/979,992, filed on Apr. 15, 2014.
(60) Provisional application No. 62/064,040, filed on Oct. 15, 2014.

(51) Int. Cl. G01N 27/447; G01N 33/561; G01N 1/40

(52) U.S. Cl. CPC G01N 27/44756; G01N 27/44721; G01N 1/4077; G01N 33/561; G01N 2001/4038

(57) ABSTRACT

The present disclosure relates to devices and methods for performing isotachophoretic concentration of analytes using a porous matrix, for example, for use in diagnostic assays such as lateral flow assays. For example, the disclosure provides a method of concentrating an analyte in a sample. The method includes providing a device comprising a porous matrix having a first fluid pathway having a first end and extending to a second end, a first electrode, and a second electrode; introducing to the first pathway a first fluid comprising a trailing electrolyte, a second fluid comprising a leading electrolyte and the analyte; and applying a voltage across the first electrode and the second electrode for a time sufficient to provide an ITP plug. As described herein, the devices and methods described herein can be used in conjunction with lateral flow assay techniques to detect and quantify a variety of biochemical and biological analytes, such as nucleic acids, proteins, cells and metabolites.

### CROSS-REFERENCE TO RELATED APPLICATIONS

This application claims the benefit of priority of U.S. Provisional Patent Application No. 61/979,992, filed Apr. 15, 2014, and U.S. Provisional Patent Application No. 62/064,040, filed Oct. 15, 2014, each of which is hereby incorporated herein by reference in its entirety.

### STATEMENT OF GOVERNMENT SUPPORT

This invention was made with government support under contract no. CBET-0747917, awarded by the National Science Foundation. The government has certain rights in the invention.

### BACKGROUND OF THE DISCLOSURE

1. Field of the Disclosure

The present disclosure relates generally to isotachophoretic devices and methods. More particularly, the present disclosure relates to devices and methods for performing isotachophoretic concentration of analytes using a porous matrix, for example, for use in diagnostic assays such as lateral flow assays.

2. Technical Background

Isotachophoresis (ITP) is a nonlinear electrophoretic technique used to preconcentrate and separate a variety of ionic compounds, ranging from small metallic ions to large biomolecules such as proteins and nucleic acids. ITP is an effective electrophoretic concentration technique, with the potential of up to a million fold concentration. In ITP, sample ions focus between leading (LE) and trailing electrolytes (TE) which have co-ions with respectively higher and lower effective electrophoretic mobilities than the sample ions. When a constant voltage or current is applied across the channel, sample ions accumulate and preconcentrate by electrophoresis into a number of contiguous zones between LE and TE zones, arranged in the order of their relative electrophoretic mobilities. Each zone has uniform characteristic concentration governed by electrophoresis conservation laws, namely the Kohlrausch regulating function (for fully ionized species) or more generally the Joving Alberty function (for all analytes, including both weakly- and strongly-ionized analytes), which calculate the adjusted concentrations of species in each zone. The adjusted concentration is the concentration each species obtains by electromigration into the zones previously occupied by species with another composition.

Depending on the initial concentration of the target sample, it can be focused (concentrated) in plateau mode or peak mode ITP. High initial sample concentrations and sufficient focusing time result in plateau mode ITP, which is characterized by distinct analyte zones each at a locally uniform concentration. At trace level concentrations, sample ions rarely form a plateau zone and operate in peak mode ITP where sample ions accumulate in a concentrated sample zone between LE and TE, which we refer to as an ITP plug. Various articles have described physics of peak mode ITP using numerical, analytical and experimental methods. Khurana & Santiago theoretically and experimentally studied sample zone dynamics in peak mode ITP in a glass capillary. They varied experimental parameters governing the sample zone dynamic independently, e.g. electrolytes concentration and applied current, to validate their analytical models and to provide a guide to experimental design and optimization of practical ITP assays. They reported, that in contrast to plateau mode ITP, there is an optimum LE concentration in peak mode ITP at which the highest concentration can be reached. They also showed that higher current densities and lower TE concentrations result in higher concentration ratios. Schwarz et al developed theoretical and numerical models to account for sample dispersion in peak mode ITP and showed non-uniform axial counter electroosmotic flow (EOF) results in sample dispersion. In addition, samples with mobility values near those of the TE or LE show greater diffusion into the TE or LE, respectively. They showed that advective dispersion caused by the non-uniform counter EOF when coupled with other sources of dispersion, can drastically reduce the ITP concentration ratio. Their models allow for fast and accurate prediction of dispersed sample distributions in ITP based on known parameters including species mobilities, electroosmotic (EO) mobility, applied current density and channel dimensions.

The growing demand for increased analytical sensitivity is challenging the current format of lateral flow immunoassay (LFIA) tests. Target biomolecules present at low concentrations require novel detection, amplification, and sample pretreatment methods to improve their limits of detection (LOD). Many attempts have been made to improve LOD in LFIA, including labeling the bioreceptors with colloidal particles, chemical and colorimetric signal amplification, enzymatic signal amplification, and using multistep sample processing in two dimensional paper networks. However, these methods require additional instrumentation, sophisticated chemical processes, and significant increase in the testing time. Fernandez-Sanchez et al. reported one of the lowest detection limits for traditional LFIA on a polyethylene-based membrane at 1 μg/L. They sandwiched prostate specific antigen (PSA) between anti-PSA antibodies immobilized on the strip and a colloidal gold anti-PSA antibody tracer. They were able to achieve low LOD by introducing an extra washing step to the assay in order to reduce the background noise, but this washing step requires further manipulation by the user after sample addition and may negatively influence the reproducibility and reliability of the device.

Electrokinetic techniques have been used for 50 years on porous thin layers and membranes to separate charged molecules of small and intermediate size, e.g. paper chromatography and paper electrophoresis. These studies use electrophoretic transport to separate and analyze chemical compounds. A recent study provided a quantitative immunoassay point of care (POC) chip with a glass porous fiber sheet, which has fabric structure similar to cellulose paper. They used EOF to drive fluorescently labeled target molecules to the capture zone and compared sensitivity of their device to conventional capillary flow based LFIA. Their method lacked specificity to particular analyte molecules in a complex matrix, required a complicated fabrication procedure and a minimum test time of 20 min, and did not achieve a LOD which is consistent with conventional lateral flow tests.

Paper substrates have been widely used to construct point-of-care lateral flow immunoassays (LFIA) diagnostic devices. Paper based microfluidic devices are robust and relatively simple to operate, compared to channel microfluidic devices, which is perhaps their greatest advantage and the reason they have reached a high level of commercial success. However, paper devices have generally not been well suited for integrated sample preparation, such as sample extraction and concentration, that is desirable in complex samples with low analyte concentrations.

Accordingly, what is needed are devices and methods that can conveniently and inexpensively provide increased ion concentrations and decreased limits of detection in analytical and other devices, especially for charged organic species such as charged biomolecules.

### SUMMARY OF THE DISCLOSURE

In one aspect, the present disclosure provides a device including
a porous matrix having a first end and a second end opposing the first end, the first end and the second end defining a first axis, the porous matrix having a first fluid pathway having a first end and extending to a second end;
a first electrode disposed adjacent the first end of the first pathway; and
a second electrode disposed adjacent the second end of the first pathway.

In another aspect, the disclosure provides a method of concentrating an analyte in a sample, the method including
providing a device as described herein, the device including
   a trailing electrolyte, disposed in the porous matrix within the first fluid pathway, the trailing electrolyte comprising an ion and a counterion;
   a leading electrolyte, disposed in the porous matrix within the first fluid pathway, the leading electrolyte comprising an ion and a counterion, the ion of the leading electrolyte having a higher effective electrophoretic mobility than the ion of the trailing electrolyte; and
   an analyte disposed in the porous matrix, the analyte comprising an analyte ion and an analyte counterion, the ion of the analyte ion having a higher effective electrophoretic mobility than the ion of the trailing electrolyte and a lower effective electrophoretic mobility than the ion of the leading electrolyte; and
applying a voltage across the first electrode and the second electrode for a time sufficient to provide an ITP plug.

In another aspect, the disclosure provides a method of concentrating an analyte in a sample, the method including
providing a device comprising
   a porous matrix having a first end and a second end opposing the first end, the first end and the second end defining a first axis, the porous matrix having a first fluid pathway having a first end and extending to a second end,
   a first electrode, and
   a second electrode;
introducing to the first pathway
   a first fluid comprising a trailing electrolyte, the trailing electrolyte comprising an ion and a counterion, the first fluid being disposed such that the first electrode is in conductive contact with the first end of the first pathway, and
   a second fluid comprising a leading electrolyte, disposed in the porous matrix within the first pathway, the leading electrolyte comprising an ion and a counterion, the ion of the leading electrolyte having a higher effective electrophoretic mobility than the ion of the trailing electrolyte, the second fluid being disposed such that the second electrode is in conductive contact with the second end of the first pathway, and
   the analyte, the analyte comprising an analyte ion and an analyte counterion, the ion of the analyte ion having a higher effective electrophoretic mobility than the ion of the trailing electrolyte and a lower effective electrophoretic mobility than the ion of the leading electrolyte; and
applying a voltage across the first electrode and the second electrode for a time sufficient to provide an ITP plug.

As described herein, the devices and methods can be used in the detection of various analytes. For example, the devices and methods described herein can be used in conjunction with lateral flow assay techniques to detect and quantify a variety of biochemical and biological analytes, such as nucleic acids, proteins, cells and metabolites.

Additional aspects and embodiments will be evident to the person of ordinary skill in the art in view of the present disclosure.

### BRIEF DESCRIPTION OF THE DRAWINGS

FIG. 1 is a schematic cross-sectional view of a device according to one embodiment of the disclosure;

FIG. 2 is a schematic cross-sectional view of a method according to one embodiment of the disclosure;

FIG. 3 is a schematic plan view of a porous matrix suitable for use in certain embodiments of the disclosure;

FIGS. 4A and 4B are perspective views of a device according to one embodiment of the disclosure, in an open state and a closed state, respectively;

FIG. 5 is a schematic cross-sectional view of a device according to one embodiment of the disclosure;

FIG. 6 is a schematic cross-sectional view of a device according to another embodiment of the disclosure;

FIG. 7 is a schematic cross-sectional view of a device according to another embodiment of the disclosure;

FIGS. 8A and 8B are schematic cross-sectional and plan views of a device according to another embodiment of the disclosure;

FIGS. 9 and 10 are schematic plan views of two porous matrices suitable for use in various embodiments of the disclosure;

FIGS. 11A and 11B are schematic plan views of a porous matrix including a detection zone having a series of separated areas of detection substance, respectively before and after analyte capture;

FIGS. 11C and 11D are graphs of probe and target concentration for two detection schemes;

FIG. 12 is a schematic comparison between a conventional lateral flow assay and an ITP-enhanced lateral flow assay of the present disclosure;

FIG. 13 is a schematic perspective view of an experimental setup for ITP experiments;

FIG. 14 is a plan view of a porous matrix used in various experiments described in the Examples;

FIG. 15 is a plan view of another porous matrix used in various experiments described in the Examples;

FIGS. 16A and 16B are normalized spatiotemporal maps of an ITP plug formed in an ITP experiment;

FIG. 17 is a graph of the effect of trailing electrode concentration on the ITP stacking ratio;

FIG. 18A is a set of five instantaneous images of ITP plug generation and migration;

FIG. 18B is a plot of stacking ratio and fraction of sample as a function of axial location of the ITP plug;

FIG. 18C is plot of width of the ITP plug vs. axial location of the ITP plug;

FIG. 19A is a graph of stacking ratio as a function of applied current across the membrane;

FIG. 19B is a graph of the fractional number of moles as a function of applied current for the same conditions as in FIG. 19A;

FIGS. 20A and 20B are, respectively, schematic views of experimental setups for an ITP-enhanced lateral flow assay and for a conventional lateral flow assay;

FIG. 20C is a set of experimental snapshots taken at 5 different times showing that ITP-focused IgG labeled with AF488 is captured at the test zone;

FIG. 21 is a plot fraction of bound probe h as a function of target concentration for ITP-LF and LF for a pair of experiments;

FIG. 22 is a set of snapshots of a paper device used for colorimetric detection of the target using both ITP-LF (left) and conventional LF (right) for the initial target concentration ranging from 0.1-15 mg/L; and

FIG. 23 is a plot of ratio of calculated limits of detection for lateral flow and ITP-enhanced lateral flow assays as a function of assay time.

### DETAILED DESCRIPTION

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice. Thus, before the disclosed processes and devices are described, it is to be understood that the aspects described herein are not limited to specific embodiments, apparati, or configurations, and as such can, of course, vary. It is also to be understood that the terminology used herein is for the purpose of describing particular aspects only and, unless specifically defined herein, is not intended to be limiting.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As will be understood by one of ordinary skill in the art, each embodiment disclosed herein can comprise, consist essentially of or consist of its particular stated element, step, ingredient or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. As used herein, a material effect would result in a statistically significant reduction in the effectiveness of a compound in treating cancer, a parasitic infection or a yeast infection.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

In various aspects and embodiments, the disclosure relates to the integration of isotachophoresis (ITP), an electrokinetic concentration and extraction technique, onto devices based on porous matrices such as membrane and paper materials (e.g., nitrocellulose-based paper) or microchannels or capillaries having disposed within a porous matrix. The disclosure demonstrates ITP to be a rapid and portable technique to improve the limit of detection of porous matrix-based assays (e.g., based on paper or membrane materials) by extracting specific molecules in a complex matrix and by increasing their concentration at the location of the detection zone.

The methods and devices described herein can be used, for example, to improve the limit of detection of various analytical techniques (e.g., thin-film, capillary or microchannel-based techniques) such as lateral flow assays. ITP has been largely used in traditional capillary-based microfluidic devices as a pre-treatment method to preconcentrate and separate variety of ionic compounds. The present disclosure demonstrates show that ITP using a porous matrix such as nitrocellulose is capable of a high level of sample concentration (e.g., up to several hundred times or even up to several thousand times) and extraction (e.g., up to 60% from 100 μL samples and more than 80% from smaller sample volumes, or even higher levels of extraction (e.g., 90%)). ITP based on porous matrices such as paper can be challenged by Joule heating and resulting evaporation when the porous matrix is open to the environment. In certain embodiments of the methods and devices described herein, high sample concentration is achieved by mitigating evaporation-induced dispersion using device structures that help to maintain the porous matrix in a substantially hydrated state. Moreover, the present disclosure demonstrates that ITP on a porous matrix (e.g., nitrocellulose membrane) can be powered and run several times by a small button battery, suggesting that it could be integrated to a portable point-of-care diagnostic device. Accordingly, the present disclosure demonstrates the potential of ITP to increase sensitivity of paper-based lateral flow assays in conditions where small analyte concentrations are present in complex biological samples.

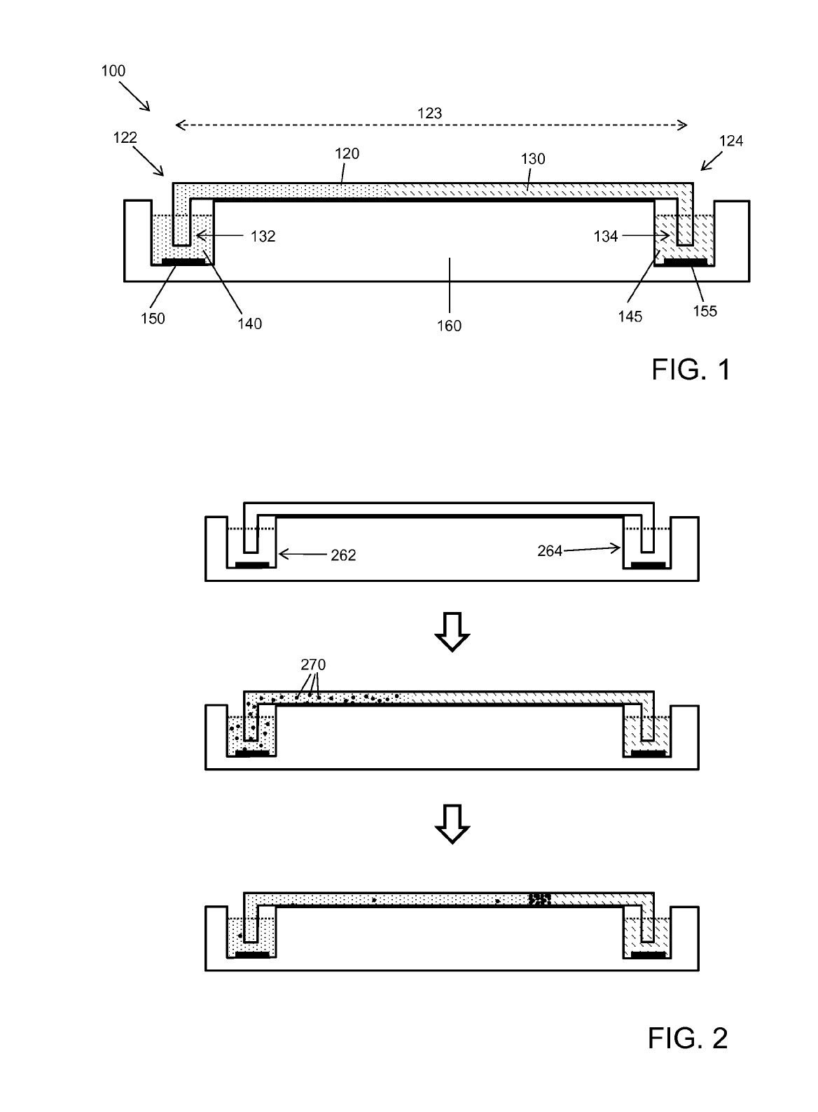

One embodiment of the disclosure is a device useful for performing concentration of an analyte via ITP. One example of such a device is shown in schematic view in FIG. 1. Device 100 includes a porous matrix 120 having a first end 122 and a second end 124 opposing the first end. The first end and the second end of the porous matrix define a first axis 123. The porous matrix includes a first fluid pathway 130 extending from a first end 132 to a second end 134. In this embodiment the first fluid pathway extends substantially along the direction of the first axis. Of course, in other embodiments, the first fluid pathway does not extend substantially along the direction of the first axis. In some embodiments, the first fluid pathway follows a circuitous path (e.g., as defined by a channel or capillary). The device also includes a first electrode 150 disposed adjacent the first end of the first pathway; and a second electrode 155 disposed adjacent the second end of the first pathway. The electrodes are positioned so that when liquids are disposed in the device as described below, the first electrode is in fluid communication with the first end of the first pathway, and the second electrode is in fluid communication with the second end of the first pathway.

In the embodiment of FIG. 1, the device further includes a first fluid 140 and a second fluid 145, disposed in the porous matrix (i.e., within the pores thereof) within the first pathway. The first fluid 140 includes a trailing electrolyte having ion and a counterion, and the second fluid 145 includes a leading electrolyte having an ion and a counterion. The ion of the leading electrolyte has a substantially higher effective electrophoretic mobility than the anion of the trailing electrolyte. The second fluid is disposed closer to the second end of the first pathway than is the first fluid. Of course, the person of ordinary skill in the art will appreciate that devices as described herein can be provided without the fluids disposed therein; in such embodiments, the user can add the fluids to the device as necessary to perform an ITP concentration as described herein.

In use, the device can further include an analyte (i.e., the substance to be concentrated via isotachophoresis) disposed in the porous matrix. The analyte includes an analyte ion and an analyte counterion. The analyte ion has a higher effective electrophoretic mobility than the ion of the trailing electrolyte and a lower effective electrophoretic mobility than the ion of the leading electrolyte. As will be described in further detail below, the analyte can initially be provided at a variety of locations along the fluid pathway (e.g., provided together with the leading electrolyte or the trailing electrolyte, or added somewhere else along the device). Upon the application of an electrical potential, the isotachophoresis process will concentrate the analyte at the interface between the leading electrolyte and the trailing electrolyte. As the person of ordinary skill in the art will appreciate, the analyte can be provided to the device in a variety of forms. For example, the analyte can provided as part of a complex sample (e.g., a bodily fluid or sample such as blood, urine, mucous, stool, food particles, cells that are harvested from a body by scraping). The devices and methods described herein can be operated by the person of ordinary skill in the art to isolate the analyte from other components of such a complex sample. The analyte can be a complex molecule, e.g., a biomolecule, and can be weakly or strongly ionized. The ionization state can depend upon the media in which it is disposed (e.g., with respect to ionic strength, pH, the presence of other ions). In certain embodiments, a particular molecule of interest is converted to an analyte ion/counterion pair by reaction, complexing or coordination with some other molecule. For example, in one embodiment, a molecule of interest (e.g., in a complex matrix such as urine or mucous) is contacted with a specific label (e.g., a complimentary protein bound to an indicator such as a nanoparticle). Such contacting can be performed, for example, in the porous matrix itself, or in one of the first or second fluids before it is added to the porous matrix. The molecule of interest can bind to the complementary protein bound to the indicator, thus forming an analyte that can be both concentrated and detected and quantified. In certain embodiments, when a molecule of interest has a very low or very high mobility, the person of ordinary skill in the art can subject that molecule to conditions to provide a desirable analyte ion/counterion for isotachophoretic concentration. For example, in embodiments in which an indicator is used as described above, the indicator can substantially alter the mobility of the complex and thus provide an analyte ion/counterion. For example, if the molecule of interest is only weakly charged (or even uncharged), it can have too low a mobility for isotachophoretic concentration (e.g., because it cannot be effectively transported by the electric field, or a trailing electrolyte is not available that has a lower mobility), The complimentary protein-indicator molecule can bind to the molecule of interest and effectively modify (in this case, by increasing) its mobility so that it can be effectively separated from the complex matrix and concentrated using ITP. Of course, the person of ordinary skill in the art can select pH and other values of the fluids that ensure the molecule of interest is a charged state necessary to be present as an analyte ion/counterion suitable for ITP concentration.

In certain embodiments, the analyte has a concentration at least about two orders, or even less than about four orders of magnitude smaller than the concentration of the leading electrolyte in the second fluid, and a concentration at least about two orders, or even less than about four orders of magnitude smaller than the concentration of the trailing electrolyte in the first fluid.

Another embodiment of the disclosure relates to a method for concentrating an analyte. The method includes providing a device as described herein, i.e., including a first fluid and a second fluid disposed in a first fluid pathway as described herein. The device also includes an analyte disposed in the porous matrix, the analyte comprising an analyte ion and an analyte counterion, the ion of the analyte ion having a higher effective electrophoretic mobility than the ion of the trailing electrolyte and a lower effective electrophoretic mobility than the ion of the leading electrolyte. The method also includes applying a voltage across the first electrode and the second electrode for a time sufficient to provide an ITP plug, as described in more detail below.

Another embodiment of the disclosure relates to a method for concentrating an analyte. The method includes providing a device including comprising a porous matrix having a first end and a second end opposing the first end, the first end and the second end defining a first axis, the porous matrix having a first fluid pathway having a first end and extending to a second end, a first electrode, and a second electrode in conductive contact with the second end of the first pathway (e.g., as described above). The device can be, for example, a device as described herein. The method also includes introducing to the first pathway (i.e., in the porous matrix) a first fluid comprising a trailing electrolyte, the trailing electrolyte comprising an ion and a counterion, the first fluid being disposed such that the first electrode is in conductive contact with the first end of the first pathway, and a second fluid comprising a leading electrolyte, disposed in the porous matrix within the first pathway, the leading electrolyte comprising an ion and a counterion, the ion of the leading electrolyte having a higher effective electrophoretic mobility than the ion of the trailing electrolyte, the second fluid being disposed such that the second electrode is in conductive contact with the second end of the first pathway, and the analyte, the analyte comprising an analyte ion and an analyte counterion, the ion of the analyte ion having a higher effective electrophoretic mobility than the ion of the trailing electrolyte and a lower effective electrophoretic mobility than the ion of the leading electrolyte. The method also includes applying a voltage across the first electrode and the second electrode for a time sufficient to provide an ITP plug (i.e., a concentrated sample disposed at the interface between the leading electrolyte and the trailing electrolyte). As the person of ordinary skill in the art will appreciate, the application of the voltage can substantially separate the analyte from other components of the matrix in which it is originally provided (e.g., blood, mucous, cell or urine components), allowing the simplification of analysis and quantification of the analyte.

An example of a method for concentrating an analyte in a sample is shown in schematic view in FIG. 2. In the method of FIG. 2, a device including a porous matrix and two electrodes as described above is first provided. Then, a first fluid and a second fluid are introduced to the first fluid pathway, here, by introducing the first fluid to the first well 262 and the second fluid to the second well 264 and allowing the fluids to wick into the porous matrix from either end thereof. In this embodiment, while the electrodes are not initially in conductive conduct with the respective ends of the first fluid pathway, the introduction of the first fluid and the second fluid makes the necessary conductive contacts. The analyte 270 is also introduced to the first fluid pathway. In the embodiment of FIG. 2, the analyte is introduced to the first fluid pathway by including it in the first fluid (e.g., before it is introduced to the device); as the person of ordinary skill in the art will appreciate, the analyte can be introduced to the first pathway in a variety of other manners. For example, the analyte can be included with the second fluid (e.g., before it is introduced to the device), or added separately to the porous matrix. After the fluids and the analyte are introduced, a voltage is applied across the first electrode and the second electrode for a time sufficient to provide an ITP plug, as described in more detail below.

In the methods and devices as described herein, a variety of porous matrices can be used by the person of ordinary skill in the art. The porous matrix can have, for example, an average pore size in the range of about 0.5 to about 10 μm, e.g., in the range of about 0.5 to about 7 μm, about 0.5 to about 5 μm, about 0.5 to about 3 μm, about 1 to about 10 μm, about 1 to about 7 μm, about 1 to about 5 μm, about 1 to about 3 μm, about 3 to about 10 μm, about 3 to about 7 μm, about 3 to about 5 μm, or about 5 to about 10 μm, or about 0.1 μm to about 100 μm, or about 0.1 μm to about 10 μm, or about 1 μm to about 100 μm, or about 10 μm to about 100 μm. The porous matrix as described herein is desirably highly porous (e.g., at least about 65% porous, at least 70% porous, at least 75% porous, at least 80% porous, at least 85% porous, or even at least 90% porous, e.g., about 65% to about 90% porous, about 65% to about 95% porous, about 65% to about 98% porous, about 70% to about 90% porous, about 70% to about 95% porous, about 70% to about 98% porous, about 75% to about 90% porous, about 75% to about 95% porous, about 75% to about 98% porous, about 80% to about 90% porous, about 80% to about 95% porous, about 80% to about 98% porous, about 85% to about 90% porous, about 85% to about 95% porous, at about 85% to about 98% porous, about 90% to about 95% porous, or about 90% to about 98% porous. In certain embodiments, the internal surface area ratio (i.e., the internal pore surface area per external apparent area) is in the range of about 50 to about 200, e.g., in the range of about 50 to about 150, about 50 to about 125, about 50 to about 100, about 75 to about 200, about 75 to about 150, about 75 to about 125, about 75 to about 100, about 100 to about 200, about 100 to about 150, or about 100 to about 125. The porous matrix for example, can be a paper or membrane material. For example, in certain embodiments, the porous matrix is formed from nitrocellulose. In other embodiments, the porous matrix is formed from glass (e.g., glass fiber), nylon (e.g., nylon fiber), polyester, polyvinylidine fluoride, polycarbonate or cellulose acetate. In certain embodiments, the porous matrix is a porous material (e.g., as described above, or filled with particulate matter or porous particulate matter) disposed within a microchannel or capillary; in such embodiments, the porous matrix can have the pore size, internal surface area ratio and/or porosity as described above.

The porous matrix can generally be formed to be relatively thin, e.g., in the range of about 100 μm to about 5 mm in thickness, e.g., about 500 μm to about 5 mm, about 1 mm to about 5 mm, about 100 μm to about 3 mm, about 500 μm to about 3 mm or about 1 mm to about 3 mm. In certain embodiments, the length of the porous matrix from its first end to its second end (e.g., along its first axis) is in the range of about 5 mm to about 200 mm, e.g., in the range of about 5 mm to about 100 mm, about 5 mm to about 75 mm, about 5 mm to about 50 mm, about 5 mm to about 40 mm, about 5 mm to about 30 mm, about 10 mm to about 200 mm, about 10 mm to about 100 mm, about 10 mm to about 75 mm, about 10 mm to about 50 mm, about 10 mm to about 40 mm, about 10 mm to about 30 mm, about 20 mm to about 200 mm, about 20 mm to about 100 mm, about 20 mm to about 75 mm, about 20 mm to about 50 mm, about 20 mm to about 40 mm, or about 20 mm to about 30 mm. In certain embodiments, the width of the porous matrix (e.g., in a direction perpendicular to the first axis or in a direction perpendicular to the direction of the first fluid pathway, for example, at the position of the detection zone as described below) is in the range of about 0.5 mm to about 30 mm, e.g., in the range of about 0.5 mm to about 25 mm, or about 0.5 mm to about 20 mm, or about 0.5 mm to about 15 mm, or about 0.5 mm to about 10 mm, or about 0.5 mm to about 5 mm, or about 1 mm to about 30 mm, or about 1 mm to about 25 mm, or about 1 mm to about 20 mm, or about 1 mm to about 15 mm, or about 1 mm to about 10 mm, or about 1 mm to about 5 mm. The porous matrix can, for example, be provided in a generally rectangular form, as described below with respect to FIG. 14.

In certain embodiments, the length of the first fluid pathway from its first end to its second end is in the range of about 5 mm to about 200 mm, e.g., in the range of about 5 mm to about 100 mm, about 5 mm to about 75 mm, about 5 mm to about 50 mm, about 5 mm to about 40 mm, about 5 mm to about 30 mm, about 10 mm to about 200 mm, about 10 mm to about 100 mm, about 10 mm to about 75 mm, about 10 mm to about 50 mm, about 10 mm to about 40 mm, about 10 mm to about 30 mm, about 20 mm to about 200 mm, about 20 mm to about 100 mm, about 20 mm to about 75 mm, about 20 mm to about 50 mm, about 20 mm to about 40 mm, or about 20 mm to about 30 mm. In certain embodiments, the width of the first fluid pathway (in a direction perpendicular to the direction of the first fluid pathway, for example, at the position of the detection zone as described below) is in the range of about 0.5 mm to about 30 mm, e.g., in the range of about 0.5 mm to about 25 mm, or about 0.5 mm to about 20 mm, or about 0.5 mm to about 15 mm, or about 0.5 mm to about 10 mm, or about 0.5 mm to about 5 mm, or about 1 mm to about 30 mm, or about 1 mm to about 25 mm, or about 1 mm to about 20 mm, or about 1 mm to about 15 mm, or about 1 mm to about 10 mm, or about 1 mm to about 5 mm.

As described above, the first fluid pathway extends from a first end to a second end within the porous matrix. The first fluid pathway need not be particularly defined within the matrix, i.e., it need not be set off from the remainder of the matrix by any particular structures. In the device of FIG. 1, the first end 132 of the fluid pathway is disposed at the first end 122 of the porous matrix, and the second end 134 of the fluid pathway is disposed at the second end 124 of the porous matrix. In other embodiments, one or both of the first end and the second end of the fluid pathway is offset from the corresponding first or second end of the porous matrix. In certain embodiments, the first fluid pathway extends in substantially the same direction as the first axis (e.g., within 30 degrees, within 20 degrees, or even within 10 degrees of the first axis). In certain embodiments, the first fluid pathway follows a circuitous path, e.g., through a microchannel or capillary; such a configuration can allow for a longer first fluid pathway in an overall smaller device size.

The devices of FIGS. 1 and 2 further include a substrate 160. The substrate 160 includes wells 162 and 164, respectively holding the first and second fluids. The ends of the porous matrix are dipped in the wells, allowing the first and second fluids to wick into the porous matrix along the first fluid pathway. Of course, the person of ordinary skill in the art will appreciate that the first and second fluids can be introduced to the first fluid pathway via a variety of structural arrangements and methods. Moreover, the person of ordinary skill in the art will appreciate that in some embodiments, no substrate is necessary; all necessary structures can be integrated onto the porous membrane or otherwise provided. For example, first and second reservoirs for the first and second fluids can be provided as separate structures, or the first and second fluids can be introduced to the first fluid pathway manually or via some other structure(s). For example, the fluid(s) and/or the analyte can be introduced to the porous matrix through a top surface thereof. Reservoirs can be provided on the top surface of the porous matrix for this purpose, as shown in FIG. 4A. A reservoir can also be provided in the form of an absorbent material (e.g., sponge or an absorbent pad) configured to absorb liquid and contact it with the porous matrix, e.g., as shown in FIG. 6. Such an absorbent material can, for example, have a recess formed therein to allow for placement of a relatively large quantity of fluid in the sponge reservoir. The absorbent material can be formed, for example, from cellulose membrane or glass fiber.

The first and second electrodes can likewise be provided in a variety of fashions. In the embodiment of FIGS. 1 and 2, the substrate bears the first and second electrodes 150 and 155. In other embodiments of the methods and devices as described herein, the first and/or second electrodes can be provided in another fashion, for example, fabricated directly in contact with the porous matrix (e.g., by being formed directly thereon, for example, by metal evaporation, screen printing or electrochemical deposition), or being positioned in contact with the porous matrix (e.g., by being clamped against the porous matrix), or being positioned in contact with a conductive fluid (e.g., the first or second fluid) that is in contact with the first fluid pathway. As the person of ordinary skill in the art will appreciate, the electrodes need be in conductive communication with either end of the first pathway at the time that the separation via application of voltage is performed.

In order to practice the isotachophoresis step, the first and second electrodes can be coupled to an electrical voltage or power source. The electrical voltage or power source can be configured to provide sufficient voltage and current for the desired isotachophoretic process. The electric voltage or power source, the first electrode, and the second electrode can be, for example, configured so that current flows along the first fluid pathway in a direction that is substantially parallel to the direction of flow of the first fluid along the first fluid pathway (e.g., within 30 degrees, within 20 degrees, or even within 10 degrees). The voltage or power source can be, for example, a constant voltage or current source. The voltage can be applied, for example, such that a constant voltage is applied across the electrodes, or a constant current passes between the electrodes. Notably, the voltage or power source need not be a plug-in source like a laboratory source; it can advantageously be based on a battery. For example, in certain embodiments, the voltage or power source includes a battery and a voltage multiplier circuit. The battery can be, for example, a portable battery like a button battery, a cylindrical battery like a AA, AAA, C or D battery, a lantern battery, or a 9V battery. Of course, other voltage or power sources can be used, e.g., a flexible battery, a capacitor, a small fuel cell, solar powered, liquid or solid pouch battery. The voltage or power source can be, for example, configured to apply between 50-2000 volts to the device and/or configured to provide a current flow between 10⁻⁶ to 3 milliamperes to the device.

The person of ordinary skill in the art will appreciate that a variety of ionic analytes can be concentrated using the methods described herein. Thus, the analyte includes an ion and a counterion. In certain embodiments, the analyte ion is an anion, and the analyte counterion is a cation. In other embodiments, the analyte ion is a cation and the analyte counterion is an anion. The person of ordinary skill in the art will appreciate that certain nonionic analyte species (e.g., amines and carboxylic acids) can be converted to ionic analytes by a judicious choice of pH of the first fluid and/or the second fluid. For example, the analyte can be a poly- or oligopeptide (e.g., a protein or a fragment thereof), a poly- or oligonucleotide or -side (e.g., a DNA or RNA or fragment thereof), an alkaloid, a metabolite, a vesicle, a metal or metal oxide nanoparticle, a nanostructure like a carbon nanotube or a fullerene, a cell, a virus, a hormone or an enzyme.

The analyte can be provided to the first fluid pathway in a variety of methods. For example, as described above, it can be provided in the first fluid. In other embodiments of the methods and devices as described herein, the analyte can be provided to the first fluid pathway at a location between the first and second ends thereof, for example, at a sample zone (which can be, e.g., defined by a window in a final packaged device). Notably, the analyte can be provided from a solution having a relatively low concentration; the devices and methods described herein can substantially concentrate the analyte to provide for improved limits of detection in an assay. For example, in certain embodiments of the methods and devices as described herein, the analyte has a concentration that is at least about three, at least about four, or even at least about five orders of magnitude smaller than the concentration of the leading electrolyte in the second fluid, and a concentration at least about three, at least about four, or even at least about five orders of magnitude smaller than the concentration of the trailing electrolyte in the first fluid (e.g., each on the order of about three to about seven, about three to about six, about three to about five, about four to about seven, about four to about six, or about five to about seven orders of magnitude (e.g., when it is disposed in the porous matrix, or when it is disposed in the first fluid or the second fluid before being introduced to the porous matrix). And as described above, the analyte can be part of a complex matrix, and/or can be generated from an insufficiently mobile species by techniques such as complexation or reaction.

The person of ordinary skill in the art will select first and second fluids and leading and trailing electrolytes in view of the particular analyte of interest. For example, the first and second fluids can be provided with a desired pH and ionic strength such that they are sufficiently conductive for isotachophoresis (e.g., on the order of 5 μS/cm to about 50 μS/cm) and that the analyte is in a desired form (e.g., a desired ionic form).

Each of the leading and trailing electrolytes includes an ion and a counterion. When the analyte ion is an anion, the leading and trailing electrolyte ions are anions, and when the analyte ion is a cation, the leading and trailing electrolyte ions are cations. The leading and trailing electrolytes are selected such that the analyte ion has a higher effective electrophoretic mobility than the ion of the trailing electrolyte and a lower effective electrophoretic mobility than the ion of the leading electrolyte. Thus, in the isotachophoretic process, the analyte ion will concentrate at the boundary between the faster-moving leading electrolyte ions and the slower-moving trailing electrolyte ions. As the person of ordinary skill in the art will appreciate, effective mobility is a function of degree of dissociation of analytes which is typically a function of their dissociation constants (e.g., Kₐ), local pH and local ionic strength. A detailed discussion on choice of an electrolyte system for ITP is provided, for example, in Everaerts, F. et al., Isotachophoresis: Theory, Instrumentation and applications; Elsevier, 1976, and Bagha, S. S. et al., Electrophoresis 2010, 31, 910-919, each of which is hereby incorporated herein by reference in its entirety. In certain embodiments of the methods and devices as described herein, the difference between the effective mobilities of the ion of the leading electrolyte and the ion of the trailing electrolyte is at least about 3 nm²V⁻¹s⁻¹, at least about 6 nm²V⁻¹s⁻¹ or even at least about 9 nm²V⁻¹s⁻¹. For example, in certain embodiments in which the analyte ion is an anion, the leading electrolyte is a strong acid, e.g., hydrochloric acid, nitric acid, phosphoric acid, or carbonic acid. In certain embodiments in which the analyte ion is an anion, the trailing electrolyte is a weak acid, e.g., HEPES, serine, glycine, trycine, TAPS, MOPS, BES, ACES, MES, or MOPSO. In certain embodiments in which the analyte ion is a cation, the leading electrolyte is a strong base, e.g., sodium hydroxide, magnesium hydroxide, calcium hydroxide, potassium hydroxide. In certain embodiments in which the analyte ion is an anion, the trailing electrolyte is a weak base, e.g., Tris, Bis-Tris, creatinine, pyridine, EACA, ammediol, ethanolamine. In certain embodiments of the methods and devices as described herein, the counterion of the trailing electrolyte is the same as the counterion of the leading electrolyte. The leading and trailing electrolytes can be at any desired concentration in the first and second fluids, respectively. For example, in certain embodiments, the concentration of the trailing electrolyte in the first fluid is between about 0.1-500 mM. In certain embodiments of the methods and devices as described herein, concentration of the leading electrolyte in the second fluid is between about 50-1000 mM.

In certain embodiments of the methods and devices as described herein, the difference between the effective mobilities of the ion of the analyte and the ion of the trailing electrolyte is at least about 1 nm²V⁻¹s⁻¹, at least about 2 nm²V⁻¹s⁻¹ or even at least about 4 nm²V⁻¹s⁻¹. In certain embodiments of the methods and devices as described herein, the difference between the effective mobilities of the ion of the analyte and the ion of the leading electrolyte is at least about 1 nm²V⁻¹s⁻¹, at least about 2 nm²V⁻¹s⁻¹ or even at least about 4 nm²V⁻¹s⁻¹.

In certain embodiments of the methods and devices as described herein, other materials can be present in the first pathway (e.g., by being provided as part of the first and/or second fluids). Such materials can, for example, reduce the effects of electroosmotic flow, and can, for example, provide more rapid analysis, improved reproducibility, and resolution, for example, by coating the pore surface of the porous matrix. The additive can be, for example, a non-ionic surfactant or a non-ionic polymer, e.g., poly(vinyl alcohol), a poly(alkylene glycol) polymer, a polyethylene glycol (PEG), polyvinylpyrrolidone (PVP), Tween-20, Triton-X, polylactams, substituted polyacrylamide derivatives, and water-soluble methylhydroxyethyl derivatives of cellulose. This additive can be present in the device in a dry state (e.g., before the electrolytes and/or the fluids are added); or can be added with fluid when performing the assay (e.g., with one or more of the electrolytes and/or the first or second fluids).

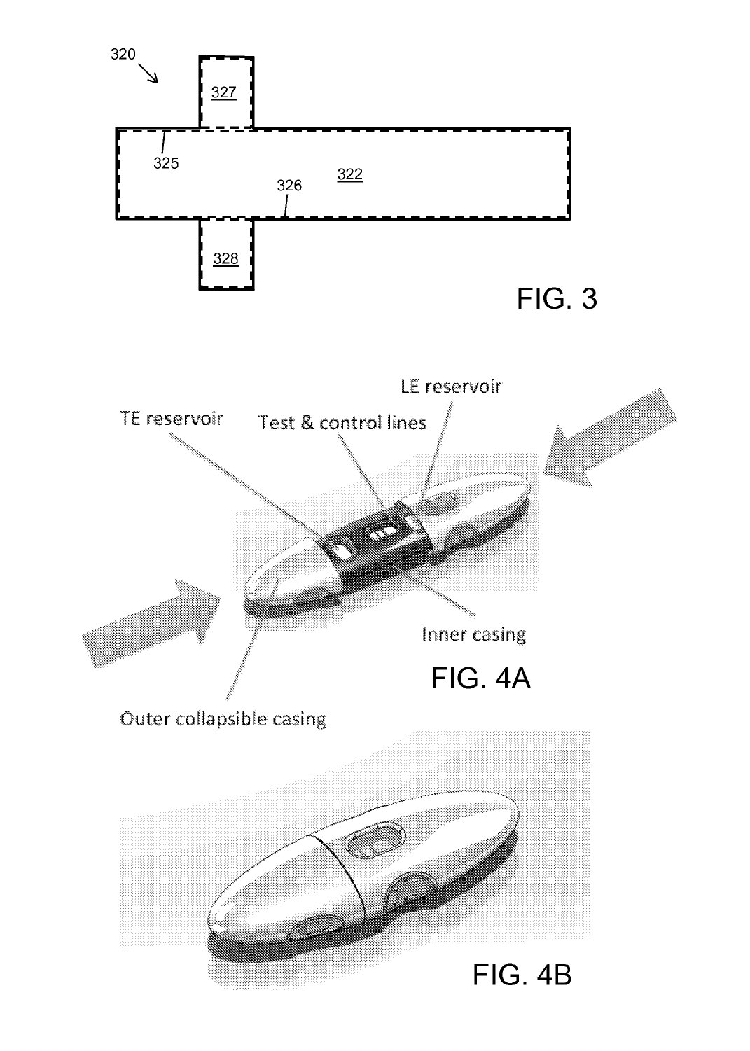

As noted above, one problem with prior art isotachophoretic methods and devices is the drying out of the electrolyte fluids, especially in configurations in which the porous matrix is open to the atmosphere. Accordingly, in one embodiment of the methods and devices as described herein, the porous matrix includes a first portion disposed generally along the first fluid pathway (e.g., along the first axis), the first portion having a first lateral edge and a second lateral edge opposing the first lateral edge, and at least one tab extending from the first lateral edge of the first portion. An example of such a porous matrix is shown in schematic plan view in FIG. 3. Porous matrix 320 includes a first portion 322 having a first lateral edge 325 and a second lateral edge 326 opposing the first lateral edge. Porous matrix 320 also includes a tab 327 that extends from the first lateral edge 325 of the first portion. The porous matrix can further include at least one tab extending from the second lateral edge; in the embodiment of FIG. 3, a tab 328 extends from second lateral edge 326. The tabs in the embodiment of FIG. 3 are formed at generally right angles to the strip-shaped first portion, forming a generally cruciform shape. Of course, in other embodiments, the one or more tabs can be formed at some other angle with the first fluid pathway (or the first axis), e.g., an angle between about 45° and about 90° to the first fluid pathway (or the first axis). The one or more tabs can have a width that is, for example, in the range of about 20% to about 150% (e.g., about 50%) of the width of the first portion. In certain embodiments, the one or more tabs are positioned in the range of about ⅕ of the way to about ½ the way (e.g., about ⅓) from the first end to the second end of the porous matrix. While in the embodiment of FIG. 3, the tabs are disposed toward the first end of the porous matrix, the person of ordinary skill in the art will understand that a tab can be placed anywhere along the porous matrix in fluid communication with the first fluid pathway.

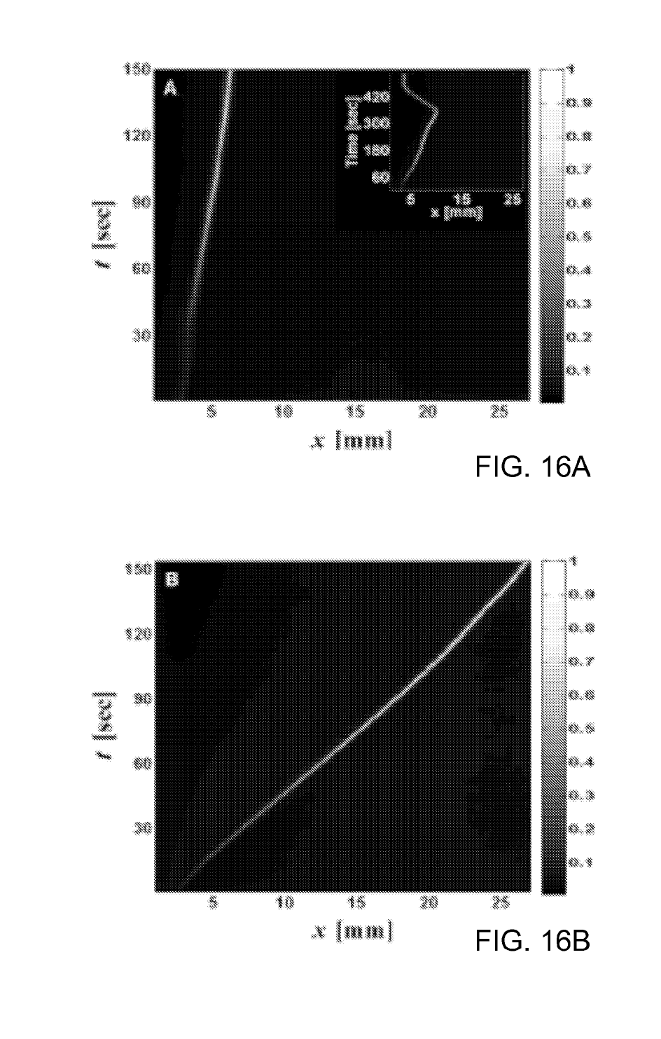

Each tab can be operatively coupled to a source of a third fluid, e.g., by being dipped into or otherwise fed by a reservoir of the third fluid. In certain embodiments of the methods described herein, the fluid is introduced into the second pathway, then from the second pathway to the first pathway (e.g., through capillary action) as the first pathway evaporates liquid due to Joule heating. The one or more tabs can thus provide a third fluid to ensure that the first pathway remains sufficiently wet for the isotachophoretic method. In certain embodiments, the third fluid is desirably substantially free of the leading electrolyte or the trailing electrolyte. In other embodiments, the third fluid includes one or more of the leading electrolyte and the trailing electrolyte. In certain embodiments, the third fluid includes or consists of water (e.g., deionized water). The third fluid can be, for example, substantially pure water, or can include additives such as buffers and polymeric species as described above. Particular embodiments are described further below with respect to FIG. 15.

In certain embodiments of the devices and methods as described herein, the device includes a substantially water-impermeable cover disposed over the porous matrix. The water-impermeable cover can also ameliorate the effects of Joule heating by trapping water in the porous matrix. The impermeable cover can, for example, be formed in contact with the porous matrix, e.g., as a coating or layer disposed thereon. In such embodiments, the substantially water-impermeable cover can have a window formed therein to allow for the application of a sample including the analyte into the first fluid pathway. In other embodiments, the substantially water-impermeable cover can be formed as part of a chamber in which the porous matrix is disposed. Such a chamber can, for example, be closed over the porous matrix at any time, e.g., either before or after the first fluid, the second fluid, and/or the analyte is disposed in the first fluid pathway. As the person of ordinary skill in the art will appreciate, all fluids described herein are desirably substantially aqueous fluids (e.g., in which the solvent is at least 85%, at least 90%, at least 95%, or even at least 99% water).

In certain embodiments, the porous matrix is disposed in a casing. The casing can, for example, closeable, such that a sample (and any of the first, second and third fluids that are not already contained within the casing) may be added then the casing closed. The casing can help to protect the reservoirs and the porous matrix from contamination and spilling, and can also protect the user from electric shock during operation. In certain embodiments, the casing is resiliently closed, so that it cannot in normal operation be reopened. The device can be configured such that the closing of the casing initiates the application of electric voltage and starts the isotachophoresis, e.g., by the closing of a switch or the contact of electrodes. In other embodiments, the isotachophoresis can be initiated by the application of one or more of the fluids to the device. For example, the device can be configured such that the application of the first liquid or the second liquid to the device can complete a circuit and thus initiate a sequence of commands by a control circuit, including the application of the electric field. The casing can also include reservoirs for any of the first second and third fluids (e.g., molded into the casing), and can include an aperture for the loading of an analyte sample onto the porous matrix in the first fluid pathway thereof, as well as a window for viewing the results of the assay (e.g., as discussed in more detail below). One example of such a device, similar in appearance to existing pregnancy tests, is shown in FIGS. 4A and 4B, in an open and closed state, respectively.

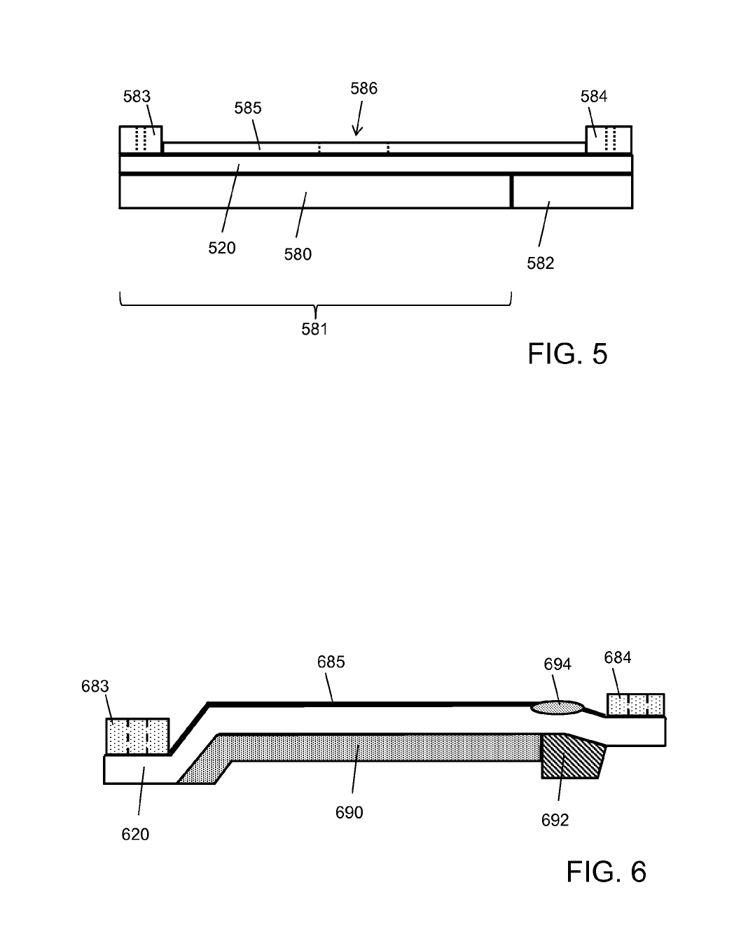

Another embodiment of the devices as described herein can be configured as a substantially flexible device. For example, in one embodiment, the device includes a flexible battery, upon which a flexible porous matrix is disposed. The first and second electrode can be operatively coupled to the battery. The device can include a flexible section in which the battery and the porous matrix are not constrained from flexing. One example of a flexible device is shown in schematic cross-sectional view in FIG. 5. In this device, a porous matrix 520 (e.g., nitrocellulose membrane) is substrate-bonded or otherwise fabricated on a flexible battery 580, such that the device has a flexible zone 581. Any type of fuel battery or fuel cell can be used as the “battery” as described herein, e.g., a flexible battery, flow battery, microfluidic fuel cell, or capacitor. In the device of FIG. 5, the battery is operatively coupled to an electronic circuit 582 configured to provide the desired voltage and current flow to the isotachophoretic process. The electronic circuit can, for example, be controlled by the user, for example, by a button, or can be activated by application of the first or second liquid. At either end, the device can include any structure as described herein for application of the first or second liquid. For example, the device can have one or more reservoirs (e.g., cup shaped) formed in fluid communication with the porous matrix for application of the first and second liquids. Alternatively, the device can include an absorbent pad, optionally with a well formed therein to serve as a location to hold a suspended drop of first or second liquid (i.e., as shown in FIG. 5 by reference numerals 583 and 584). In such devices, the electrodes might be embedded in the absorbent pads or be physically placed inside the punched reservoir in the absorbent pad. The analyte can be added, for example, as part of the first liquid (i.e., with the trailing electrolyte), or through a separate spot on the porous matrix (not shown in FIG. 5). The device of FIG. 5 also includes a water-impermeable cover 585 disposed over the porous matrix. The cover can include a window or opening 586 formed therein for viewing the results of the assay (as described in more detail below).

Another example of a device is shown in cross-sectional view in FIG. 6. This embodiment is built on a flexible battery 690; amplifier and microcontroller 692 can be actuated by the user by flexible switch 694 (as is commonly used in electronic devices). The electronic components as described herein, can, for example, be fabricated on a flexible substrate. The porous matrix 620 is disposed on the flexible battery; absorbent pads 683 and 684 can be used to apply first and second fluids to the device. In this embodiment, the device includes a substantially water-impermeable layer 685 on top of the porous matrix to minimize water loss.

The devices described herein can be used in a number of different ways. As described above, the devices described herein can include reservoirs, wells, or open areas of porous matrix into which a user can put the necessary fluids. In other embodiments, the devices can be operated via a dipping technique, in which the ends of the device are sequentially dipped into the desired fluids, and capillary action draws the fluids into the porous matrix. Thus, the first liquid and the second liquid can be provided in separate containers into which the user can dip the ends of the device. The analyte can be added to the first liquid, so that it is introduced to the device together with the trailing electrolyte. Alternatively, the analyte can be separately placed on the device (e.g., before introduction of the first and second fluid), for example, using the methods described above.

For example, in one embodiment, a method includes dipping one end of the device into the second liquid (containing the leading electrolyte) such that it contacts the second end of the first fluid pathway. It can be dipped for a time necessary to draw sufficient second liquid into the porous matrix; this time will depend on the details of the structures, but can be, for example, in the range of 1-10 seconds, or about 3 seconds. Then, the other end of the device can be dipped into the first fluid (containing the leading electrolyte) such that it contacts the first end of the first fluid pathway. Here, too, the dipping can be performed for a time necessary to draw sufficient first liquid into the porous matrix; this time will depend on the details of the structures, but can be, for example, in the range of 1-10 seconds, or about 3 seconds. The liquids or the containers in which they are provided can be color-coded, to be matched with colored ends of the device in order to ensure the correct fluid is contacted with the correct part of the device. After dipping, the electronics can be engaged, e.g., by the user using a button formed on the device or by the electronic circuit itself via a switch that is activated by the addition of the first fluid. The user can read the device after a given time, e.g., in the range of 1-10 minutes. In embodiments in which the first and/or second fluids are provided by dipping, there need not be a reservoir or absorbant pad formed at the respective end of the porous matrix; as long as the matrix can be exposed to the fluid, capillary action can draw the fluid into the matrix.

The use of incorrect fluids (e.g., either improperly formed, or confusing first fluid for second fluid) is a possible failure mechanism for the devices and methods described herein. In certain embodiments, the first fluid and/or the second fluid are formulated onboard the device. The device can include, for example, the dry material of the trailing electrolyte and/or the leading electrolyte disposed in one or more zones thereof (e.g., as a solid, for example, disposed in the porous matrix or in a well), such that the user can simply add water (or some other aqueous solution) to the device (e.g., using any of the methods described above) and the respective electrolyte dissolves into the water to form the respective fluid. The respective electrolytes can be provided, for example, in a reservoir coupled to the respective end of the first fluid pathway, or can be provided dispersed in the porous matrix itself. Electrolyte materials can be packaged for the user (e.g., in blister packs), desirably in proper strength for the measurement, in concentrated form or as a dissolvable solid.

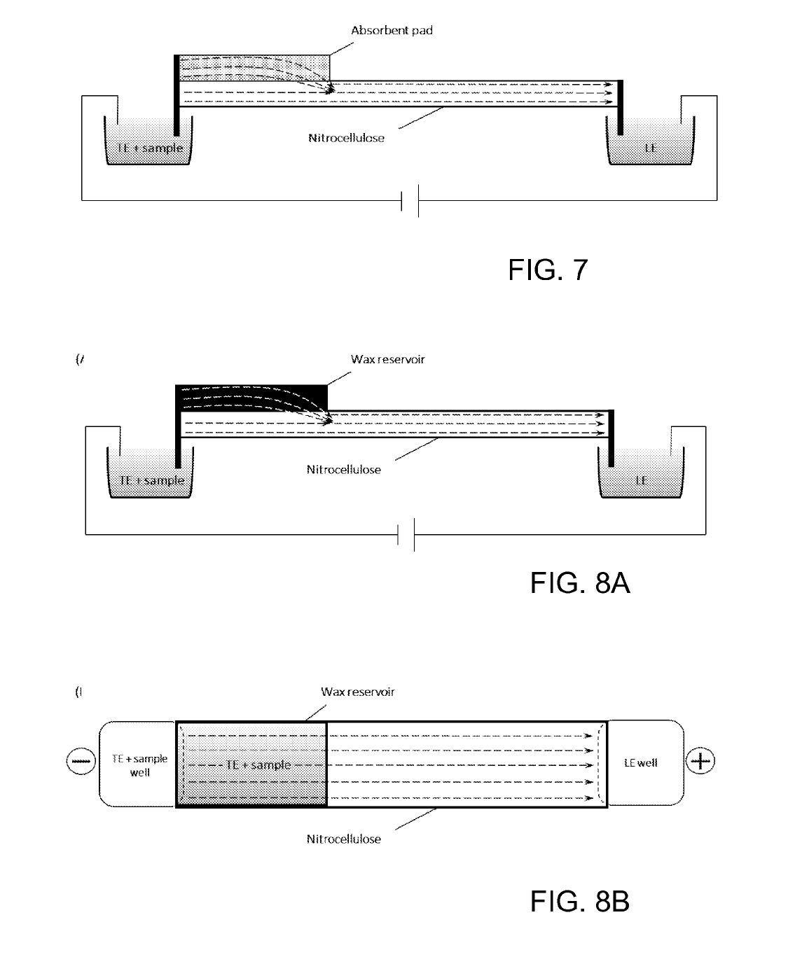

Real-world samples are often provided as exceedingly dilute solutions. Accordingly, in certain embodiments, it can be desirable to provide a relatively high amount of sample to the isotachophoresis device or method. Described herein are various methods and structures that can be used by the person of ordinary skill in the art to provide relatively large sample sizes (for example, in excess of about 100 μL, e.g., up to about 300 μL, up to about 500 μL, or even up to about 1 mL) to the isotachophoretic devices and methods described herein. For example, in one embodiment, an absorbent pad is disposed on the porous matrix at the first end of the fluid pathway, the absorbent pad being in fluid communication with the first pathway, as shown in cross-sectional schematic view in FIG. 7. Dashed lines with arrows represent the electric field lines. The high porosity and void volume of the absorbent pad can allow for addition of a large sample volume to the device. With respect to each of the fluids, a device can include, for example, only an absorbent pad (e.g., as shown in FIG. 6), only a reservoir, or both an absorbent pad and a reservoir. In another embodiment, a reservoir can be constructed (e.g., from a wax or a polymer) on the top surface of the porous matrix at the first end of the first fluid pathway, the reservoir being in fluid communication with the first pathway. The reservoir can have an opening (e.g., at the top thereof) to allow for addition of sample. Wax printing is widely used on paper-microfluidic devices to construct channels and barriers in a simple way and with low cost; such methods can be adapted for use here. Moreover, the wax reservoir can be easily sealed by the casing of the test kit and may allow addition of more sample compared to use of an absorbent pad. A device including a wax reservoir is shown in schematic cross-sectional view in FIG. 8A and in schematic plan view in FIG. 8B

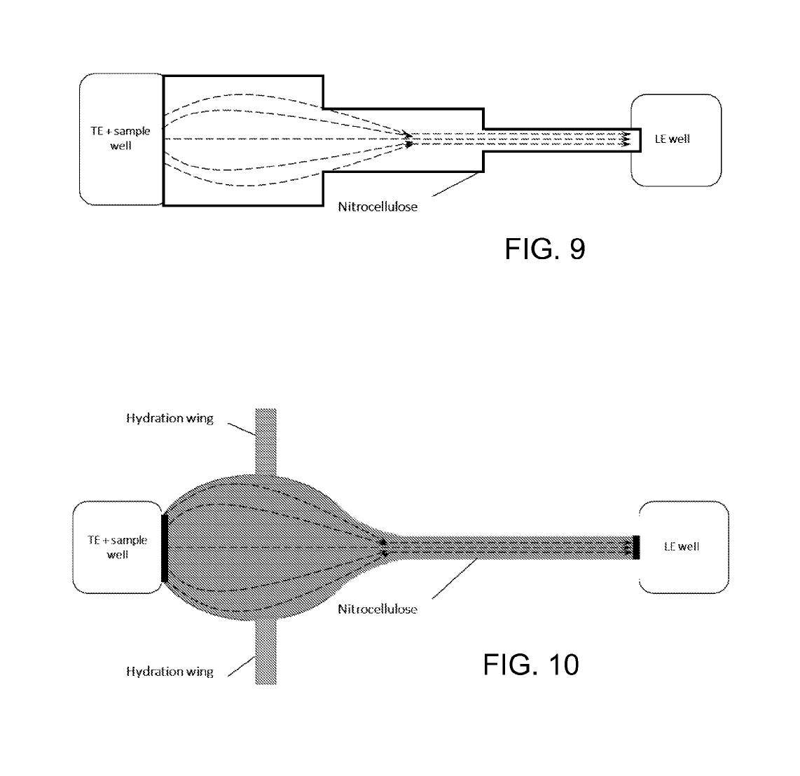

Another approach to increase the sample volume is to increase the surface area of the porous matrix at the first end of the first fluid pathway (e.g., at the first end of the porous matrix). Thus, the area in which analyte is exposed to the electric field is larger, thereby allowing more analyte to be concentrated in the ITP plug. Accordingly, in certain embodiments, a first portion of porous matrix has a first zone and a second zone, the first zone being disposed more toward the first end of the porous matrix than the second zone, the first zone having a substantially greater average width than the second zone (i.e., excluding any tabs extending from the first portion thereof). For example, the width of the first zone can be in the range of about 1.5 to about 10 times the width of the second zone. Desirably, the ITP plug is present in the second zone during operation of the device. FIGS. 9 and 10 are schematic plan views of two examples of porous matrices, in which dashed lines with arrows represent the electric field lines. The person of ordinary skill in the art will appreciate that the absorbent pad and reservoirs described with respect to FIGS. 7, 8A and 8B can advantageously be used in embodiments with increases surface area at the first end of the first fluid pathway. Moreover, as shown in FIG. 10, it can be desirable to include one or more tabs (e.g., as described above with respect to FIG. 3) in order to help mitigate evaporation. In other embodiments (e.g., when the analyte is added on the leading electrolyte side of the device), the first zone is disposed more toward the second end of the porous matrix than the second zone; the device can otherwise be as described above.

As the person of ordinary skill in the art will appreciate, the devices and methods described herein can be used to perform a variety of assays. Generally, the porous matrix can include in the first fluid pathway a detection zone. The detection zone can be configured to provide a variety of types of measurements. For example, the detection zone can include a detection substance that will interact (e.g., via reaction, binding or coordination) with the analyte to provide some detectable change, e.g., a color change, chemi- or bioluminscence or a darkening, or a change that is detectable using any of a variety of instrumental techniques, e.g., colorimetric, spectrophotometric, fluorescence or electrochemical detection). The detection substance can be, for example, anchored to the material of the porous matrix (e.g., through chemical bonding), as would be apparent to the person of ordinary skill in the art. Of course, in other embodiments, the detection substance is not anchored to the material of the porous matrix. The detection substance can be, for example, a small molecule, a colorimetric reagent, a nanoparticle, an oligo- or polypeptide (e.g., a protein), or an oligo- or polynucleotide. The detection zone can thus be configured from an assay such as an immunoassay, an ELISA, a sandwich assay or a nucleotide binding assay. In other embodiments, no particular detection substance need be present; in such embodiments, a variety of techniques can be used to detect the analyte (e.g., colorimetric, spectrophotometric, fluorescence or electrochemical detection) The methods described herein can thus include contacting the ITP plug with the detection zone. The person of ordinary skill in the art will appreciate that virtually any detection scheme useful in lateral flow assays or microfluidic-based assays can be applied to the present devices.

Thus, the methods described herein can include transmitting the ITP plug to the detection zone, and detecting the analyte in the detection zone. In certain embodiments, the device can include an optical readout system configured to perform an optical measurement on the detection zone; and the method can include performing an optical measurement on the detection zone after contacting the ITP plug therewith.

For example, assay techniques such as immunoassays, sandwich assays and competitive binding assays can be used to identify the presence and amount of an analyte biomolecule. In one such embodiment, a species that binds the analyte (e.g., a complimentary or anti-biomolecule) is deposited on porous matrix in the detection zone. The analyte biomolecule binds to an indicator that has a binding species (e.g., a complimentary or anti-biomolecule) on one side and an indicator species on the other side (e.g., metal or polymer nanoparticle, fluorescence label, enzyme that induces chemiluminesence). The indicator could be mixed with the analyte or one of the electrolytes so these are already bound when they are being concentrated by ITP. The concentrated analyte bio-molecule that is bound to the indicator reacts with the binding species in the detection zone, and in this way forms a “sandwich” on the membrane. In certain embodiments, the indicator species is visible; in such cases, a visible line or spot formed in the detection zone indicates presence of analyte. In other embodiments, the indicator species is not visible as a colorimetric line, but instead can be detected via fluorescence or chemiluminesence. The person of ordinary skill in the art will adapt other detection schemes for use with ITP-concentrated material. For example, DNA can be detected using DNA hybridization assays, with an immobilized oligonucleotide in the detection zone. Other methods of detection can be used, such as electrochemical or capacitive detection (e.g. field effect). For example, the detection zone can include one or more electrodes that are functionalized with specific biomolecules; or one or more electrodes for the performance of cyclic voltammetry to detect the presence of analyte through some IV curve signature.

As the person of ordinary skill in the art will appreciate, the detection zone can also include a control substance, configured to provide a detectable change when the assay is performed even in the absence of analyte, and thus provide a control to the assay. Thus, in certain embodiments, the control substance and the detection substance can be provided adjacent to one another in the detection zone, with a detectable change at the site of the control substance being indicative of the assay working correctly.

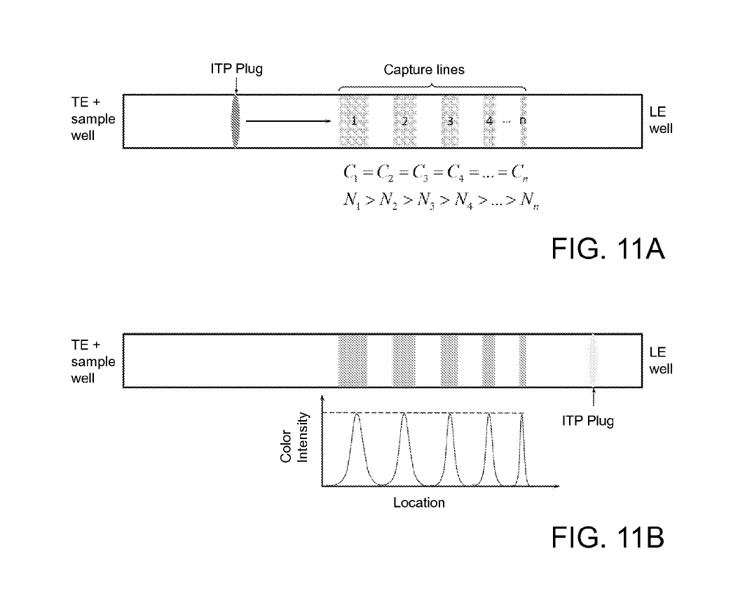

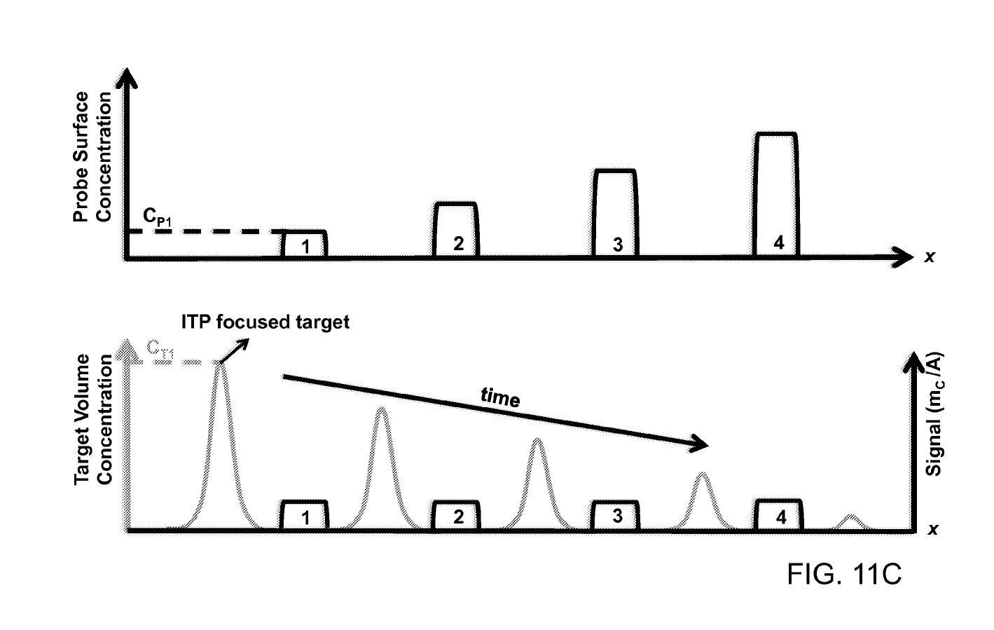

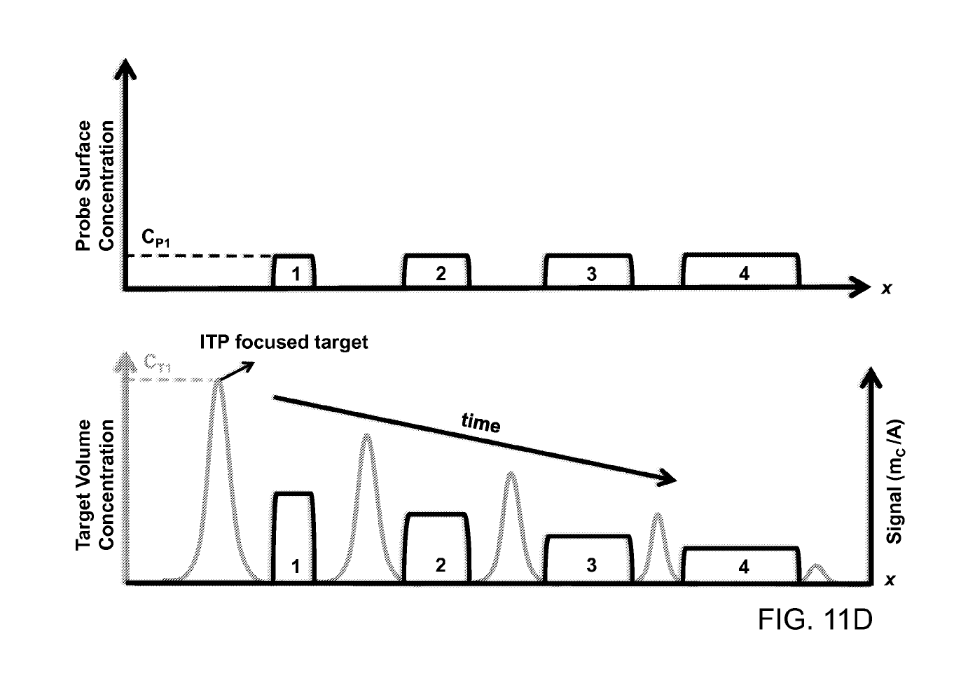

Although qualitative detection may be suitable for some detection scenarios, like a pregnancy test, many clinical decisions for medical diagnosis require a more quantitative determination of the concentration of an analyte (e.g., a biomarker related to a disease state). To provide semi-quantitative assay results, in certain embodiments of the devices and methods described herein, the detection zone can include a series of separated areas of detection substance. One such embodiment is shown in schematic plan view in FIGS. 11A and 11B.

Here, a series of immobilized capture reagent lines are used to quantify the amount of analyte in the sample. As the ITP plug containing highly concentrated analyte molecules migrates across the device, binding of the analyte to the capture reagent results in generation of multiple colored lines. Accordingly, concentrations of the analyte can then be easily calculated by knowing the number of colored lines, surface concentration of the immobilized capture reagent, and the binding ratio of the analyte/capture reagent. This calibration can be performed just once; an end-user can simply multiply the number of colored band to a constant value provided by the manufacturer to calculate the amount of analyte in the sample. To generate the same color intensity for each line, i.e. surface concentration of the probe, the width of each line and number of moles of capture reagent can be deposited, as shown in FIG. 11. In FIG. 11A, the ITP plug containing highly concentrated analyte is formed and travels toward the test lines, In FIG. 11B, the ITP plug sweeps over the test lines resulting in generation of the color bands which is directly proportional to the initial amount of target in the sample. Capture reagent lines have different widths and amount of reagent, N, so that the color intensity of each line, which directly correlates to the reagent surface concentration (C), would be the same, representing known amount of analyte captured. In certain embodiments, the lines can the same width but the surface concentration of the capture reagent can start off small and increase with assay distance, in order to compensate for decreasing concentration of the ITP plug as it traverses the lines. For example, each line may represent an original target concentration of 1 nanomole/mL of analyte; three lines would then represent 3 nanomoles/mL of analyte concentration. Of course, in other embodiments, different calibration techniques can be used to relate the number of visible lines to the analyte concentration.

FIGS. 11C and 11D show two examples of detection schemes. In the scheme of FIG. 11C, the size of the lines of capture reagent are the same, but they have increased concentrations of capture reagent moving to the right of the device, so as to capture roughly similar amounts of analyte from the diminishing amount of analyte as it flows from left to right. In the scheme of FIG. 11D, the concentration of capture reagent is the same among the stripes, but the stripes farther right are wider, so as to more efficiently capture analyte as its amount diminishes as it flows from left to right.

In other embodiments, the detection zone can include a plurality of sections, each of which is configured to target a different analyte. For example, the detection zone can include a plurality of proteins immobilized in separate sections thereof, to allow for multiplexed detection. For example, a single test can be used to detect a plurality of biomarkers (e.g., those related to chlamydia and gonorrhea).

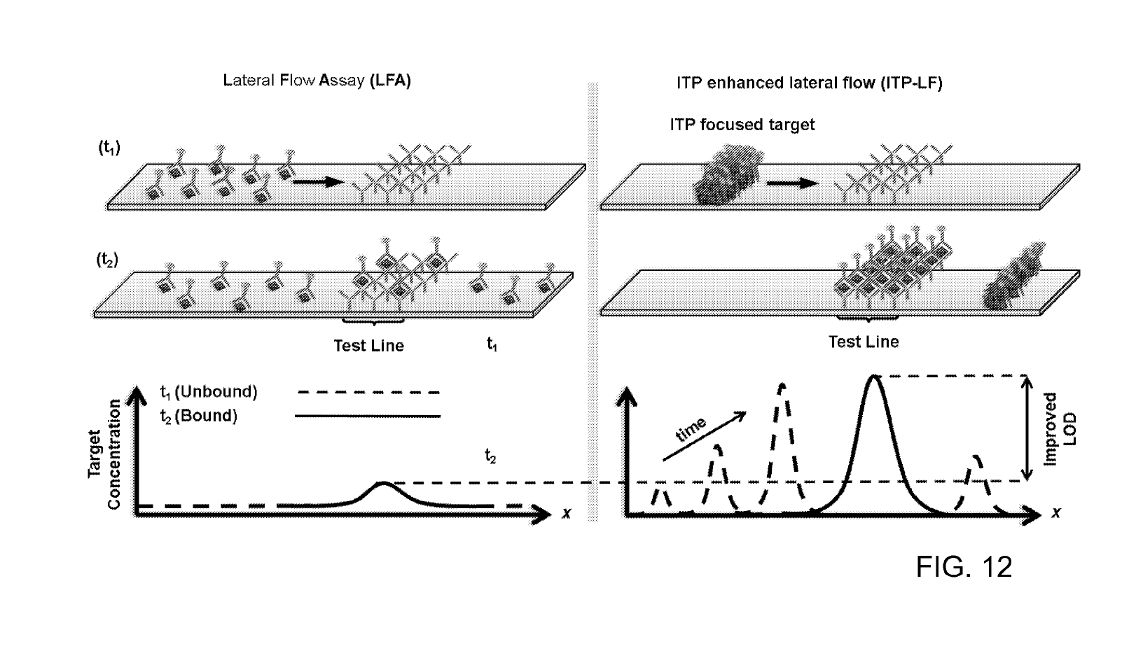

FIG. 12 demonstrates how the addition of ITP can improve lateral flow assays. As shown in the diagram, the limit of detection wan be greatly improved in the ITP-enhanced lateral flow assay at the right side of the figure, as compared to the conventional lateral flow assay shown in the left side of the figure.

Various aspects and embodiments of the disclosure are further described with respect to the following non-limiting Examples.

### EXAMPLES

The experiments described here demonstrate the use of peak mode ITP on nitrocellulose membranes with the goal of improving the limit of detection of paper-based linear flow assays. As demonstrated here, ITP on nitrocellulose has the potential to target specific molecules in a complex matrix and increase their concentration at a test zone. It also has the capacity to operate with relatively large volume of sample, e.g., up to 100 μL, to extract a large fraction of the sample even when the analyte of interest is dilute in a relatively large-volume. Nitrocellulose membrane was used to conduct the ITP experiments described here because it has been widely applied to lateral flow assays and paper-based microfluidic devices. The structure and porosity of nitrocellulose membrane are highly controllable, it has a high contrast background for colorimetric assays, it is inexpensive, and it has high binding capacity for bio-molecules. Several studies regarding the design and optimization of ITP conditions and chemistry are described. A quantitative analysis of the ITP sample concentration is also provided by reporting stacking ratio and amount of sample accumulate in the sample zone. Here, stacking ratio is defined as the ratio of sample concentration in the plug to its initial concentration in the TE reservoir. Also described is the use of a cross-shaped membrane geometry, which results in larger ITP stacking ratios by mitigating sample dispersion induced by evaporation from the membrane free surface and Joule heating. Finally, a portable, watch-battery powered electronic circuit is used to perform ITP on nitrocellulose membranes with performance consistent with a regulated bench-top power supply, demonstrating that portable ITP devices can be made using porous matrices. Various experiments are described in B. Y. Moghadam et al., “Isotachophoretic Preconcentration on Paper-Based Microfluidic Devices,” Anal. Chem. 86, 5829-37 (2014), and B. Y. Moghadam et al., “Two Orders of Magnitude Improvement in Detection Limit of Lateral Flow Assays Using Isotachophoresis,” Anal. Chem., 87(2), 1009-1017 (2015), each of which is hereby incorporated herein by reference in its entirety, including all supplemental information.

### Isotachophoretic Concentration Experiments

### Experimental Setup and Protocols

### Materials

A series of anionic ITP experiments were performed to the capability of this technique in the concentration of a target sample on nitrocellulose membrane. The anionic dye Alexa Fluor 488 succinimidyl ester (Molecular Probes, Eugene, Oreg.) was used as the sample; leading and trailing electrolytes were based on the selection of this sample. AF488 was used as a sample because of its extensive use in conventional ITP assays and its exceptional optical stability at a wide range of pHs. The leading electrolyte (LE) consisted of 40 mM HCl (i.e., with fast-moving chloride anions), in 80 mM Tris buffer. The trailing electrolyte (TE) was 10 mM HEPES (i.e., with slow-moving 4-(2-hydroxyethyl)-1-piperazineethanesulfonate ions), in 20 mM Tris buffer, mixed with 50 nM of sample. The buffering counter ionic species and the pH of the LE and TE electrolytes were selected in such a way that maximal differences in effective electrophoretic mobilities could be obtained. As the person of ordinary skill in the art will appreciate, effective mobility is a function of degree of dissociation of analytes which is typically a function of their dissociation constants (e.g., Ka), local pH and local ionic strength. Tris was selected as the counter ion and buffering agent since it is positively charged, and it has strong buffering capacity at pH=8 due to its pKa value which does not exceed more than 1 pH units from that of the electrolytes. Total ionic strength, (and thus conductivity) of the TE was selected to be lower than of the LE in order to increase the flow of sample ions into the ITP plug, as will be discussed in detail below. A detailed discussion on choice of an electrolyte system for ITP is provided, for example, in Everaerts, F. et al., Isotachophoresis: Theory, Instrumentation and applications; Elsevier, 1976, and Bagha, S. S. et al., Electrophoresis 2010, 31, 910-919, each of which is hereby incorporated herein by reference in its entirety. 3% Polyvinylpyrrolidone (PVP) was added to the LE to suppress counter electroosmotic flow. All chemicals were obtained from Sigma-Aldrich (St. Louis, Mo.) unless mentioned otherwise. All aqueous samples were prepared using water ultra-purified with a Milli-Q Advantage A10 system (Millipore Corp., Billerica, Mass.).

### Instrumentation

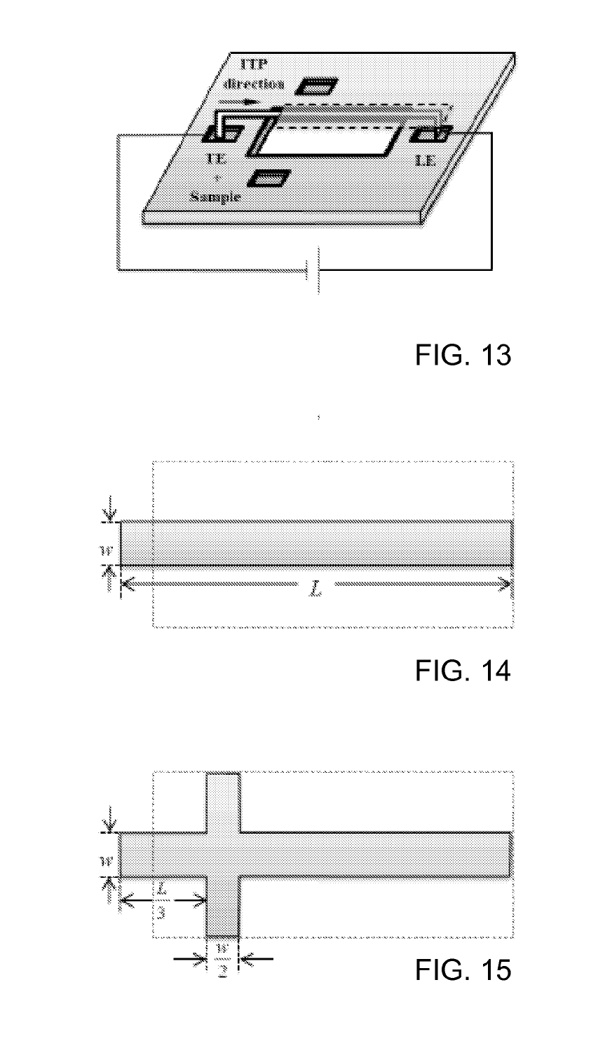

Quantitative fluorescence imaging was performed to visualize anionic dye focused by steady-state ITP experiments using a Nikon AZ100 microscope equipped with 0.5× (NA 0.05) and 5× (NA 0.5) magnification objectives (Nikon Corporation, Tokyo, Japan), a epifluorescence filter cube (488 nm excitation, 518 nm emission, Omega Optics, Brattleboro, Vt.), and a 16-bit, cooled CCD camera (Cascade 512B, Photometrics, Tucson, Ariz.). FIG. 13 shows a schematic of the ITP experimental setup. A TE reservoir (at left) and an LE reservoir (at right) were etched into the acrylic chip. A high voltage power supply (HSV488 6000D LabSmith Inc., Livermore, Vt.) applied a constant electric current. The direction of anionic ITP is from the negative electrode on the TE side to the positive electrode on the LE side. Digital processing and analysis of the data, images and movies was performed by a custom code written in MATLAB (MathWorks Inc., Natick, Mass.).

Paper-based devices were fabricated from backed nitrocellulose membrane (HF-135, Millipore, Billerica, Mass.) cut by a CO₂ laser (Universal Laser Systems, Scottsdale, Ariz.). FIG. 13 shows the overall device setup, based on an acrylic substrate having four wells formed therein. FIGS. 14 and 15 show two structures used in the experiments described herein. FIG. 14 shows a simple straight membrane; such membranes were 3.5 m in width (w) and varied in length (L=35, 40 and 45 mm). The cross-shaped structures shown in FIG. 15 have a width along the main axis of 3.5 mm, and a width of the crossbar that is that is half of the width of the main portion. As described in further detail below, the cross-shaped designs can mitigate membrane drying and the resultant decrease in concentration due to dispersion. The width of the cross wings was selected to help reduce diffusion of sample ions into the wings. The location of the cross wings was selected to be at L13 from the TE reservoir, close to the location where drying typically starts. Laser cut acrylic sample holders, with four 100 μL reservoirs, held the paper devices on a microscope stage, as shown in FIG. 13. The “west” and “east” ends (i.e., at left and right, respectively, as shown) of the membranes were folded and dipped into the TE and LE electrolyte reservoir, respectively. For the cross-shaped strips, the “north” and “south” cross wings (i.e., respectively at top and bottom as shown) were folded and dipped into reservoirs filled with DI water to provide moisture by capillary action to the membrane during the ITP experiments. Platinum wires, dipped in the acrylic reservoirs, conveyed the applied voltage to the paper devices as shown in FIG. 13.

### ITP Protocol

The reservoirs and platinum wires were rinsed with DI water several times before starting the experiments to reduce any contamination. The nitrocellulose side of the membrane was wet by putting few drops of LE from the LE side so that three fourths of the paper was wet. This wetting procedure reduces the time for the LE to wet the membrane by the capillary flow. The membrane was placed on the chip with backing side facing up and the folded ends dipped in the west (negative) and east (positive) reservoirs. The east reservoir was already filled with LE whereas 100 μL of the TE mixed with the sample was added to the west reservoir after the membrane is placed on the chip. The membrane wicks the solution as the TE reservoir is filled with the electrolyte. After establishing an interface between the TE and LE, constant electric current is applied across the two reservoirs. AF488 ions have an effective mobility intermediate between the mobility of the chloride ions of the leading electrolyte and the 4-(2-hydroxyethyl)-1-piperazineethanesulfonate ions of the trailing electrolyte. Thus, AF488 ions race ahead of the TE ions but cannot overtake the LE ions, and so they focus at the LE/TE interface. We acquired images of the concentrated sample zone (ITP plug) as it generates and travels from the TE reservoir toward the LE reservoir in a field of view that covers 27 mm of the membrane upstream of the LE reservoir (dashed lines in FIGS. 13-15).

Calibration experiments were performed before and after running each set of experiments. Fluorescence intensity of the membrane fully wetted by the homogeneous concentration of dye was measured at three different molar concentrations of 10 μM, 25 μM, and 50 μM. In the ITP experiments, the sample concentration, Cₛₐₘₚₗₑ, is calculated as

$${C}_{Sample} = \frac{{C}_{dye}}{{I}_{dye} - {I}_{back}} ( {I}_{sample} - {I}_{back} )$$

where Iₛₐₘₚₗₑ is the fluorescence intensity of the stacked sample, I_{dye} is the signal intensity corresponding to a known concentration of highly concentrated dye, C_{dye}, and I_{back} is the background intensity measured when the membrane is fully wetted by the buffer, i.e. zero dye concentration. In practice, C_{dye}/(I_{dye}−I_{back}) is the slope of the linear regression fit to the calibration points using the least square method.

Captured images from the ITP plug were width averaged (transverse, y direction) and fit with an Gaussian distribution. In peak mode ITP, the concentration profile of sample zone is approximately Gaussian rather than plateau shaped, which is governed by a local Taylor-Aris-type dispersion. The experimentally measured concentrations agreed well with Gaussian distributions. The Gaussian fits were used to determine the reported maximum intensity of the sample in the plug. An example of sample zone fluorescence intensity distribution and Gaussian fit is provided as supplementary information to Anal. Chem. 86, 5829-37 (2014).

### Results and Discussion

Concentration and accumulated moles of the sample in the sample zone are critical figures of merit in determining ITP signal strength and sensitivity and are governed by several parameters including the chemistry of the electrolytes, applied electric field, and different sources of sample dispersion. In order to achieve higher sample stacking ratios, we determined how varying these parameters along with the length of the device affect the ITP concentration on nitrocellulose membrane.

### Plug Formation and Effect of Counter Electroosmotic Flow (EOF)

FIGS. 16A and 16B show normalized spatiotemporal maps of the ITP plug as the axial location on the x-axis changes with time on the y-axis, for experiments without and with 3% PVP in the LE, respectively. In these experiments, the TE is 20 mM HEPES, 40 mM Tris; 50 nM AF488 provided therein. The LE is 40 mM HCl and 80 mM Tris. The applied current is 500 μA. These plots are generated by averaging fluorescence intensity of the plug in the transverse direction for a single ITP experiment. Net migration velocity of the ITP plug as it travels from the TE side (on left) toward the LE side (on right) can be found by calculating the slope of the line in these maps, v=dx dt. These plots show how a highly dispersed cloud of sample ions appears on the membrane at the boundary of LE/TE after applying the electric field at t=0. As the concentration of the cloud increases a plug with sharp boundaries forms and travels downstream towards the LE reservoir. Videos of the ITP plug formation and migration are available as supplementary information to Anal. Chem. 86, 5829-37 (2014) which is hereby incorporated herein by reference in its entirety. The plug migration is due to a combination of electromigration and counter electroosmotic flow. Without PVP the plug velocity slows and reverses direction toward the TE reservoir because the opposing EOF velocity becomes larger than the plug electromigration velocity, as shown in the inset of FIG. 16A. By adding PVP to the LE, as shown in FIG. 16A, the velocity of the plug increases significantly indicating that the EOF is being suppressed, resulting in a higher stacking ratio and reduction of time needed for each experiment. Axial counter EOF is a source of convective dispersion of the sample in isotachophoretic focusing which has a significant impact on the ITP concentration. The detailed quantitative analysis presented in the supplemental information to Anal. Chem. 86, 5829-37 (2014) is consistent with the experimental observation that addition of 3% PVP to the LE sufficiently decreases EOF-induced sample dispersion resulting in higher stacking ratios and increases the ITP plug velocity. Accordingly, in the remainder of the experiments described herein, 3% PVP is present in the LE.

### Effect of Electrolyte Chemistry

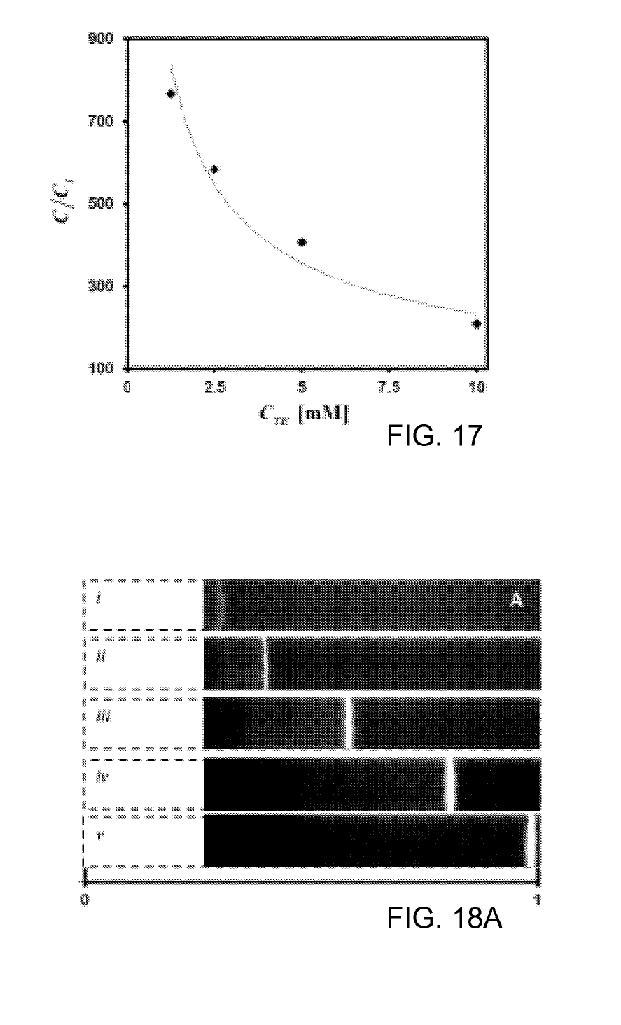

Stacking ratio and accumulation of sample ions in the ITP plug are highly affected by the concentration, (and thus the conductivity), of the trailing and leading electrolytes. An experimental parametric study was performed focusing on the concentrations of TE and LE to empirically optimize the sample stacking ratio. FIG. 17 shows the effect of TE (HEPES) concentration, C_{TE}, on the ITP stacking ratio, C/Cᵢ. Each data point represents three measurements, and the error bars denote a 95% confidence interval. The maximum dye concentration in the plug, C, was determined from the maximum of the Gaussian distribution fitted to the width-averaged intensity data. C_{TE} was varied from 1.25 mM to 10 mM and the pH of the TE was kept constant at 8.1 by adding Tris buffer at twice the concentration of HEPES. Here, the sample concentration in the TE reservoir, Cᵢ, was 50 nM and composition of the LE was fixed at 40 mM HCl, 80 mM Tris and 3% PVP. A constant current of 500 μA was applied across a 45 mm long membrane and the fluorescent signal was detected when the centerline of the plug reached 5 mm upstream of the LE reservoir. The data shows that the stacking ratio decreases with increasing concentration of the TE by 4 fold over the range tested. Since the current in the system must be conserved, increasing the concentration of the TE reduces the local electric field in the TE zone and thus results in lower flux of sample ions into the ITP plug. A power function was fit to the data in FIG. 17, C/Cᵢ=961.32C_{TE}^{-0.62} (R₂=0.96). Analytical models describing physics of peak mode ITP suggest that the stacking ratio is generally inversely proportional to the TE concentration. Without intending to be bound by theory, this discrepancy between these models and the present experimental observations are attributed to the effect of convective dispersion, which was not accounted for in those models.

At constant current density, a low concentration TE solution has lower conductivity which results in higher electric field in the TE zone, E_{TE}, due to Ohm's law and the roughly linear dependence of the solution conductivity with the ionic concentration. Higher E_{TE} increases the accumulation rate of the sample ions, dNₛ/dt, in the plug which leads to higher sample stacking. This rate equals net electrophoretic flux of sample ions from the TE zone to the sample zone, and can be expressed as:

$$\frac{{dN}_{S}}{dt} = ( {μ}_{S}^{TE} - {μ}_{TE} ) {E}_{TE} {C}_{S}^{TE}$$

where μ^{s}_{TE} is electrophoretic mobility of sample ions in the TE zone, and is the concentration of sample ions in the TE zone. This equation correctly predicts that the stacking ratio can be increased, at constant current density, by increasing the E_{TE}, i.e. lowering the C_{TE}. However, in the experiments described herein the ITP experiments could not be successfully run at TE concentrations lower than 1.25 mM due to generation of high electric field in the TE zone which burns the membrane thus results in an open circuit and termination of ITP. Higher electric fields initiate excessive Joule heating, which, on nitrocellulose membrane, can trigger sample evaporation since its surface is open to the ambient air. Excessive sample evaporation results in drying and ultimately burning of the membrane. As discussed below, drying of the membrane resulted from evaporation should be avoided since it can reduce the stacking ratio.

The dependence of the stacking ratio on the LE concentration was studied by varying the HCl concentration, C_{LE}. The TE composition was fixed at 2.5 mM HEPES and 5 mM Tris and the current density constant was fixed at 500 μA. The experiments demonstrated that, for this system, varying the LE concentration does not have a significant effect on the ITP stacking ratio (data are not shown here); this correlates well with reports in the literature. However, the person or ordinary skill in the art will appreciate that there are concentration limits that should be considered in designing the LE which are provided in the supplemental information to Anal. Chem. 86, 5829-37 (2014) In the goal of achieving LE and TE concentrations that result in highest sample concentration along with lowest heat generation-induced drying, which causes poor stacking ratios, a LE composition at 40 mM HCl and 80 mM Tris and a TE composition of 2.5 mM HEPES and 5 mM Tris are used as standard solutions throughout the remainder of the experiments described herein.

Sample Zone Characteristics Change with Distance

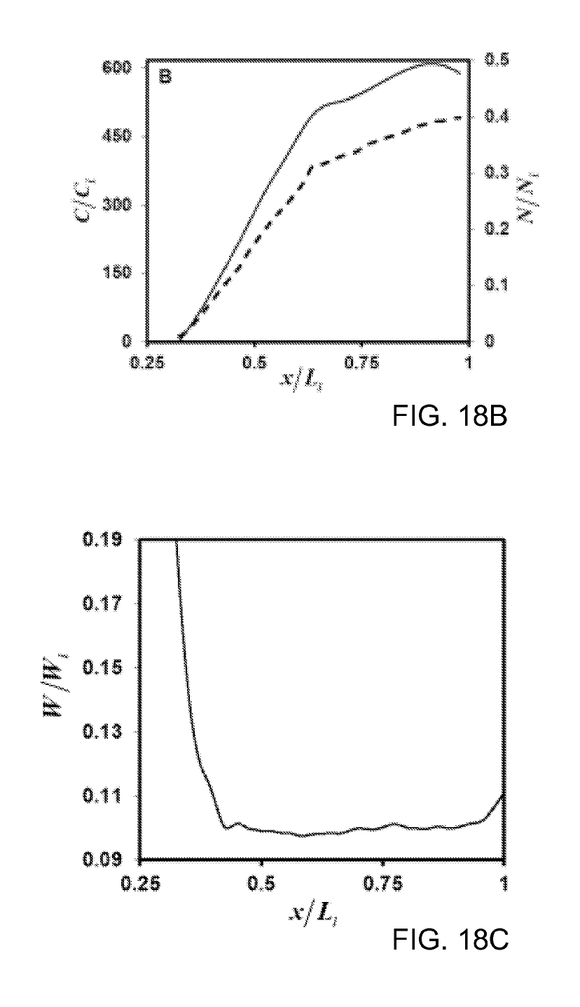

FIGS. 18A and 18B demonstrate how the shape of the plug and stacking ratio change with location. Presented here are results for a single representative ITP run on a 40 mm long membrane applied to a 500 μA constant current with the standard LE and TE solutions described above. FIG. 18A shows five instantaneous images of the ITP plug generation and migration from left to right toward the LE reservoir at i) 5, ii) 30, iii) 60, iv) 90, and v) 140 seconds after applying the electric field. The location of the plug, x, was normalized by the total length of the membrane, Lᵢ, where x/Lᵢ=0 is the TE reservoir and x/Lᵢ=1 is the LE reservoir. The field of view in FIG. 18A is x/Lᵢ=0.25-1. The sample zone concentration increases as it travels through the membrane due to the electrophoretic influx of sample ions from the TE zone into the plug. FIG. 18Ai shows the plug forming as a diffuse cloud of sample molecules. At this point plug is highly dispersed on the TE side (this is difficult to visualize with the current image settings) and exhibits a curvature in the direction of the ITP flow (toward right) which could be associated with the generation of pressure gradient because of non-uniform axial EOF. At the middle of the membrane, FIG. 18Aiii, the plug becomes upright with no curvature and then obtains a slight inverse curvature, towards the left, close to the LE reservoir as shown in FIG. 18Av. Similar sample zone curvature trends have been observed in glass capillaries by other workers; this behavior was ascribed to the internal pressure gradient generation in the capillary due to the axial mismatch of the EOF in the LE and TE zones. Note that the magnitude of the internal pressure generated in nitrocellulose membranes is not equivalent to that in capillaries because the membrane has a porous structure that is everywhere open to atmospheric pressure. However, nitrocellulose membranes are hydrophilic and thus will support some Laplace pressure. This pressure in nitrocellulose membrane is calculated to be 6 kPa (presented in the supplemental information to Anal. Chem. 86, 5829-37 (2014) which has the potential to support EOF-induced internally generated pressure gradients.

FIG. 18B provides a plot of the stacking ratio, C/Cᵢ, and fractional number of moles (number of moles of sample in the plug divided by the initial number of moles in the TE reservoir, x/Lᵢ) as a function of the axial location of the ITP plug. The stacking ratio increases linearly with distance until it reaches 500 at x/Lᵢ=0.6. The increase in stacking is a combination of a gradual increase in the number of sample moles, dashed line in FIG. 18B, and a sharp decrease in the width of the plug, as shown in FIG. 18C. Further downstream, x/Lᵢ>0.6, the rate of increase in the stacking ratio decreases. It has been shown theoretically and experimentally that this stacking rate remains constant through the entire peak mode ITP in a glass capillary. This discrepancy is attributed to convective sample dispersion caused by the strong presence of counter EOF which may have not been fully suppressed by PVP.

FIG. 18C shows the variation of the plug width normalized by the width of the membrane (3.5 mm), as a function of the plug location. The data are from a single ITP experiment in which TE was 2.5 mM HEPES/5 mM Tris with 50 nM AF488 added; LE was 40 mM HCl/80 mM Tris with 3% PVP added; the applied current is 500 μA and the length of the membrane is 40 mM. The reported width of the plug is the full width at quarter maximum of the sample zone intensity distribution. At the beginning of the ITP experiment the sample zone is wide and highly dispersed, x/Lᵢ=0.3, w/wᵢ=0.19, because a large number of sample ions are entering the membrane as a diffuse cloud. When the ITP plug forms with sharp boundaries at x/Lᵢ=0.4, the plug width decreases rapidly by 40% reaching the value of w/wᵢ=0.1 and shows a slight increase toward the LE reservoir. Previous workers have shown that at constant applied current and zero EO mobility, the width of the plug is independent of the axial location. When considerable dispersion is present the plug shows strong dispersive broadening near the TE-reservoir-region at x/Lᵢ<0.5, and depending on the absolute value of the EO mobility stays nearly unchanged or slightly increases afterwards, which correlates well with our observations in FIGS. 18C and 18Ai.

### Effect of Applied Current and Length of the Membrane

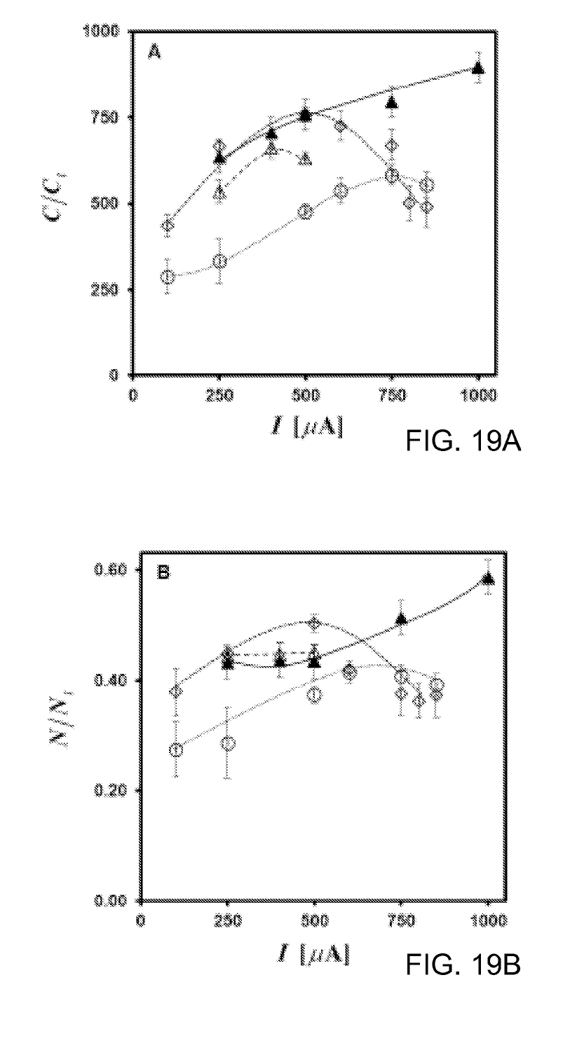

As described above, the stacking ratio and the amount of sample in plug change with electrolytes concentration and the location of the ITP plug. The experiments described here investigate how electric field and length of the membrane influence these focusing parameters. ITP experiments were performed with the applied currents ranging from 100-850 μA on membranes with varying lengths of 35, 40, and 45 mm using the standard LE and TE solutions described above. For each experiment the stacking ratio and the fractional number of moles at a point 5 mm upstream the LE reservoir were measured. FIG. 19A shows the stacking ratio as a function of applied current across the membrane. The open circles represent 35 mm long straight membranes, the open diamonds represent 40 mm long straight membranes, the open triangles represent 45 mm long straight membranes, and the closed triangles represent 45 mm long cross-shaped membranes (i.e., as described above). Each data point represents an average of at least four realizations and the error bars denote 95% confidence interval; lines are provided to allow the reader to follow the datasets. For the 35 mm long membrane, the stacking ratio increases with current and reaches the maximum value of 580 at 750 μA. By further increasing the current the stacking ratio remains nearly constant, based on a Student's t-test analysis. Higher current density results in higher local electric field in each zone which increases the counter EOF. Dispersion induced by EOF lowers the stacking ratio by reducing accumulation of sample ions and broadening the sample zone. A detailed study of the width of the ITP plug as a function of the current, provided in the supplemental information to Anal. Chem. 86, 5829-37 (2014), showed that for a 40 mm membrane the width of the plug decreases linearly until 500 μA and then increases due to dispersion that is a result of Joule heating induced evaporation. As a result, the stacking ratio plateaus at higher current densities.

These data are in a good agreement with previous theoretical predictions that sample stacking ratio increases linearly with current density in peak mode ITP. Previous studies have also demonstrated that the stacking ratio plateaus at higher current densities when the effect of dispersion becomes significant. Note that, at a given condition, the time required to complete an ITP experiment reduces by increasing the applied current. For example, the time needed for ITP experiment on 35 mm membrane at 100 μA is approximately 720 seconds while it reduces to 110 seconds at 600 μA.

When the length of the membrane was increased to 40 mm, an increase in the maximum stacking ratio to 760 at 500 μA was observed. In a longer membrane, more sample ions accumulate in the plug because they have more time to reach the sample zone resulting in higher stacking ratio. However, in the 40 mm membrane, the stacking ratio decreases at currents higher than 600 μA because of the dominant effect of dispersion caused by sample evaporation. Joule heating, which is directly proportional to the applied current, triggers evaporation of the solutions due to the open surface of nitrocellulose and at higher currents leads to a dry membrane. Drying, which is more prominent in the low conductivity TE zone, has the potential to disturb influx of sample ions into the ITP plug resulting in lower stacking ratio. Moreover, dry regions of the membrane have higher electrical resistance thus local electric field increases in order for the current to be conserved resulting in higher sample dispersion.

In order to further investigate effect of length on the stacking ratio, the length was increased to 45 mm, however, no increase in stacking ratio was observed as compared to the 40 mm membrane. This can be explained by high electrical resistant of the 45 mm long membrane which increases electric field across the membrane and thus in each ITP zone. Higher electric field in ITP zones increases dispersion due to EOF and results in a lower stacking ratio. Similar to what was observed for 40 mm membrane, currents in excess of 500 μA reduce the stacking ratio as a result of dispersion due to evaporation. Note that for 45 mm, membrane evaporation starts at lower currents, ˜500 μA, compared to 40 mm long membrane because of its higher resistance.

FIG. 19B shows the fractional number of moles as a function of applied current for the same conditions as in FIG. 19A. The fractional number of moles is an indication of amount of amount of sample extracted from the TE reservoir and focused in the ITP plug. The fractional number of moles follows roughly the same trend as the stacking ratio. For each length tested, there is a region where the fractional number of moles increases linearly with current and then reduces at higher currents due to the effect of different sources of dispersion, i.e. non-uniform EOF and evaporation. Analytical and experimental studies on peak mode ITP suggests that fractional number of moles of sample does not depend on the applied current, but these models do not account for the convective dispersion due to counter EOF which cannot be fully suppressed in nitrocellulose membranes, and were conducted in glass capillaries which is exempt from sample evaporation due to heating. FIG. 19B shows that increasing length of the membrane from 35 mm to 40 mm improves the fractional number of moles by 10% while further increasing the length to 45 mm results in an inverse trend due to dominant effect of dispersion. A 760 fold stacking ratio and sample extraction of 50% was achieved by applying 500 μA current on a 40 mm long membrane.

This data suggests that there is an optimum in both applied current and device length in respect to the maximum stacking ratio. Higher applied current leads to higher stacking ratio, more sample extraction and faster concentration. Increasing the membrane length is also desirable since it improves the stacking ratio and fractional number of moles. However, there is a limit to increasing these parameters to avoid negative impacts of sample dispersion due to evaporation and EOF. In order to obtain highest sample concentration one should find optimum length and current based on the physiochemical properties of the device, e.g. membrane type, target molecules, time constraint of the experiments.

In an effort to reach higher sample extractions, experiments were performed in which 5 μL of the sample was introduced on the middle of the membrane between the TE and LE; this can easily be done using an open surface paper based device. The measurements (not shown here) demonstrate that 80% of sample molecules can be extracted and focused using this technique.

### Novel Membrane Designs

As described herein, the sample dispersion due to the membrane drying reduces the ITP stacking ratio and fractional moles of sample. When an electric current, I, flows through an electrolyte solution with conductivity σ in a channel with cross section A, Joule heating is produced and for the electric power dissipated in a volume unit it scales as I²/Aσ. Joule heating is the main limitation in attempts to accelerate different electrophoretic analysis, including ITP, by using high currents or high voltages. Joule heating generates temperature gradients in the ITP zones which results in some inhomogeneous physical and chemical properties, e.g., mobility, pH, density, etc. Further, it can cause sample evaporation on the open surface of the nitrocellulose membrane which at higher currents and longer device length results in drying. Membrane drying disturbs electrophoretic migration of the ions and results in lower ITP concentration ratios. Therefore, excessive evaporation should be avoided in order to be able to apply higher currents across the membrane and achieve higher stacking ratio and sample extraction.

A cross-shaped membrane design, as shown in FIG. 15, can be used to mitigate the drying effects of evaporation and increase the stacking ratio of ITP. Without intending to be bound by theory, it is hypothesized that by wetting the membrane through the two added wings, the hydration of the membrane can be maintained so the sample molecules can migrate electrophoretically through the membrane. The wings are disposed closer to the TE side because enhanced drying was observed in the TE zone due to its lower conductivity. In FIGS. 19A and 19B stacking ratio and fractional number of moles are compared in experiments run in 45 mm long cross-shaped membrane (closed triangles) to a similar straight membrane (45 mm). This data shows that higher currents can be used to provide a significant increase in the stacking ratio and fraction of moles using the cross shaped membrane. At 500 μA, the stacking increases by 17% compared to the straight 45 mm membrane and reaches 750 fold. It was possible to apply currents up to 1 mA across the cross-shaped membrane and reach a stacking ratio of 900 fold. However, in this system, further increasing the current resulted in rapid and severe membrane drying and subsequent reduction in the stacking ratio. The fraction of moles accumulated follows similar trends as the stacking ratio showing that nearly 60% of initial sample can be extracted at 1 mA. Accordingly, by using cross-shaped membrane structures, it is possible to run ITP at higher currents which improves the stacking ratio, and sample extraction. Moreover, ITP can be run at much shorter times, less than 90 seconds, by operating at these high currents.

### Battery Powered ITP

One of the main drivers behind the development of paper based microfluidic devices is their relative low cost and simplicity compared to microfabricated channel devices. To demonstrate the possibility of an instrument-free device, ITP experiments were performed using commercially available button style and AA batteries and miniature voltage multiplier circuits in the place of research grade high voltage power supplies. The use of portable and inexpensive power supplies allows the integration of ITP into point-of-care diagnostics.

Using the laboratory systems described above, a stacking ratio of 760 was measured on a straight 40 mm long membrane, with the application of 500 μA of current at 440 V for 4 minutes from a laboratory power supply. This ITP experiment required 220 mW of power and consumes 40 joules (or 9 mAh) of energy. A typical smart phone battery stores 4300 mAh of energy, an alkaline AA battery stores 2122 mAh, and typical silver oxide button battery stores 170-200 mAh, each of which is several times more than the power required for the ITP experiment. Accordingly, ITP experiments were performed on a 40 mm nitrocellulose membrane powered by an AA alkaline battery. Application of a constant voltage of 300 volts across the membrane resulted in a 640 stacking ratio. The ITP assay was also powered by a single silver oxide watch battery, resulting in an average stacking ratio of 450 and extraction of 42% of sample molecules. The watch battery assay was performed 16 times on a single battery, consistent with predictions based on the battery capacity.

### Summary and Concluding Remarks

Thus, these examples demonstrate concentration of Alexa Fluor 488 on nitrocellulose membrane using peak mode ITP. Up to 900 fold increase in the initial sample concentration and 60% extraction from 100 μL of the sample were achieved by introducing a novel cross-shaped membranes that reduces sample evaporation at high currents. As a relatively large volume of sample can be used compared to the sample volumes used in microfluidic devices, ITP on paper may be especially useful in applications where the analyte of interest is dilute. Experiments are described relating to how variations in different parameters including chemistry of the electrolytes, applied current, and length of the device affect the ITP stacking ratio. For example, low TE concentration, high current density and long membrane length result in higher stacking ratio and sample extraction. Moreover the experiments described herein demonstrate that ITP on nitrocellulose membrane can be powered and run several times by a small button battery, suggesting that ITP could be integrated into a portable point-of-care diagnostic test.

### Lateral Flow Assay Experiments

### Material and Methods

### Reagents and the Electrolytes System

We performed a series of quantitative and qualitative ITP experiments on nitrocellulose membranes demonstrating the improvement of LF assay LOD using ITP. We used IgG secondary antibodies as the target and capture reagents since they are extensively used in LF assays as the capturing and labeling reagents. Moreover, these antibodies have high binding affinity for nitrocellulose membrane therefore higher surface concentration of the immobilized probe can be achieved which improves the capturing. For target analyte we used goat anti-rabbit IgG labeled with Alexa Fluor 488 fluorescence dye (CAS#2628-2-8, Molecular Probes, Eugene, Oreg.), for quantitative detection, and goat anti-mouse IgG labeled with 40 nm colloidal gold (Arista Biologicals, Allentown, Pa.) for colorimetric detection. We used bovine anti-goat IgG (Jackson ImmunoResearch Laboratories, West Grove, Pa.) as the capturing reagent, i.e. probe, since it recognizes and binds with the goat IgG and has minimal cross reactivity for bovine albumin serum (BSA) that we used to block the membrane.

To design the ITP electrolyte system, we first measured the effective electrophoretic mobility of the target IgG in a wide range of pH (data shown in Supplemental Information of Anal. Chem., 87(2), 1009-1017 (2015), which is hereby incorporated herein by reference in its entirety). IgG antibodies are large molecules of about 150 kDa thus they show low electrophoretic mobilities especially around the neutral pH, due to their isoelectric point. Based on the measured effective mobility of IgG as 6.3 nm²/Vs (at pH 7.4) we designed the TE composition as 10 mM Glycine, with the effective mobility of 0.4 nm²/Vs, buffered with Bis-Tris to pH 7.4 (σ=3 pS/cm). At this pH the binding reaction occurs at a biologically relevant condition and the difference between the mobilities of target and TE ions is maximized, which results in more potent sample focusing. We used semi-finite sample injection by mixing the TE with target analyte at different concentrations. The LE contained 40 mM HCl buffered with Tris to pH 8.1 to maintain high buffering capacity of the Tris (σ=4.2 mS/cm). The conductivity, σ, ratio of the LE and TE was maximized to 1400 to achieve the highest concentration while minimizing the Joule heating. We added 0.5% PVP to the LE to suppress electroosmotic flow. All the reagents were purchased from Sigma Aldrich (St. Louis, Mo.) unless mentioned otherwise. All aqueous samples were prepared using water ultrapurified with a Milli-Q Advantage A10 system (Millipore Corp., Billerica, Mass.).

### Paper Device Preparation

We used backed nitrocellulose membrane (HF-125, Millipore Corp., Billerica, Mass.) cut with a CO₂ laser ablator (Universal Laser Systems, Scottsdale, Ariz.) to fabricate the immunoassay devices used in both LF and ITP-LF experiments. Paper device dimensions were chosen as 40 mm×3.5 mm to maximize the target accumulation in the ITP zone while reducing the Joule heating caused by high electrical resistance of the device. We immobilized the capture reagent on the membrane by using a custom-made protein spotting system. To maintain physiological condition, capture IgG was suspended in 0.25 M NaCl and 0.01 M sodium phosphate, buffered to pH 7.0. This pH is close to the isoelectric point of the IgG, which helps to destabilize the probes in aqueous form therefore they bind more effectively with the membrane surface. The test line was located one-third of the device length away from the LE side where higher sample concentration ratio is reached. We spotted the test line with the width of approximately 800 μm and measured probe surface density of C_{P0}=2×10⁻⁹ moles per internal surface area of the membrane (0.5 fmole of probe per test line). Thickness of the test line was chosen to be in the same order of the ITP plug width, δ_{ITP}, to increase the surface interaction and C_{P0} was chosen close to the IgG binding capacity of the nitrocellulose reported by Fridley et al.'s measurements.²⁰ After spotting, the membranes were allowed to dry in room temperature for 5 minutes then further dried at 37° C. for 1 hour. To minimize the non-specific interaction of the target antibody with the membrane, we used 1% BSA as the non-reactive blocking agent.

### LF and ITP-LF Protocol

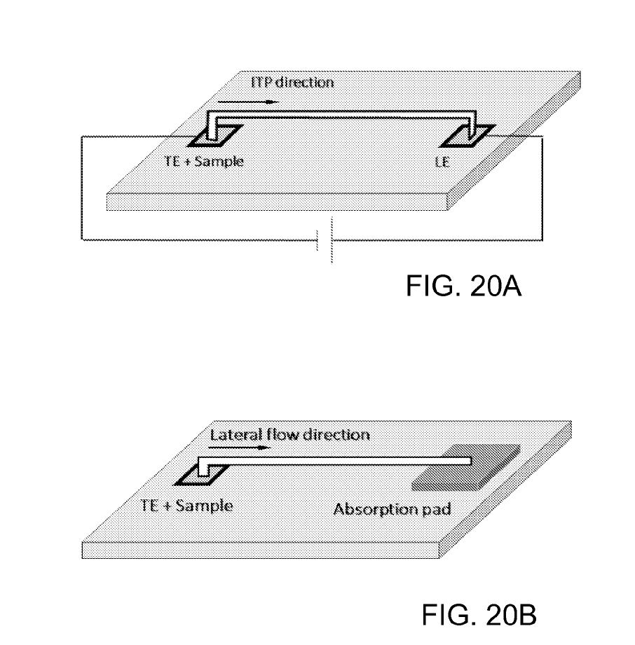

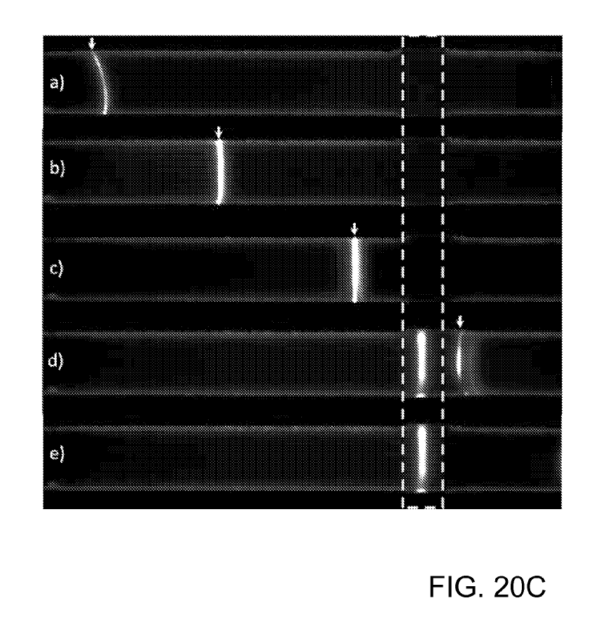

FIGS. 20A and 20B show the experimental setup for (A) ITP-enhanced LF and (B) conventional LF assay. For both assays a 40 mm long nitrocellulose membrane is used as the paper device. In ITP-LF targets migrate electrophoretically under the high electric field of 500 μA and accumulate at the narrow interface of the TE and LE. In LF assay targets migrate through the device by the capillary action. High wicking properties of the absorption pad guarantees continuous flow of the sample. FIG. 20C provides experimental snapshots taken at 5 different times showing that ITP-focused IgG labeled with AF488 is captured at the test zone. (a-c) ITP plug forms and migrates on the membrane, (d) current is reduced to 50 μA while the ITP zone crosses the test zone to increase the reaction time (e) after the plug is passed the test zone we see a specific fluorescence signal is generated. The white arrow indicates instant location of the ITP plug.

FIG. 20A shows our ITP experimental setup where the paper device is placed in an acrylic holder with the test zone closer to the LE reservoir. Before each ITP experiment the membrane was hydrated with the LE and placed on the holder with the left and right reservoirs filled respectively with 100 μL of the TE, mixed with the sample, and LE. To apply electric field across the membrane we placed the positive and negative platinum wire electrodes in the LE and TE reservoirs, respectively. We initiated the ITP-LF assay by applying 500 μA constant current to the TE reservoir and grounding the LE using a high voltage power supply (HSV488 6000D, LabSmith Inc., Livermore, Vt.). Higher currents result in a higher concentration ratio and a shorter assay time, but lower the signal strength at the test zone because the ITP zone residence time over the test line is decreased. Therefore, as the ITP zone arrives at the test zone we reduced the current to 50 μA which reduces the ITP velocity thus increases the reaction time. Plug velocity, and therefore the ITP zone residence time, is inversely proportional to the applied current. For example, in our system, t_{IPT}=9 sec at 500 μA, and t_{IPT}=284 sec at 50 μA. After the ITP plug crosses the test zone we increased the current back to 500 μA until the plug reaches the LE reservoir. Progression of the ITP plug downstream subsequently acts as an electrokinetic wash step where unbound targets are removed from the probe spots and collected at the migrating ITP plug so no further washing step is required. For the conditions mentioned above, each ITP experiment takes about 7 minutes to complete.

For conventional LF assays we used a holder with one reservoir, as shown in FIG. 20B. We placed one end of the membrane in the reservoir and the other end on a cellulose absorbent pad (HF-125, Millipore Corp., Billerica, Mass.) to wick the sample during the assay. To initiate the LF assay we filled the reservoir with the target solution mixed with TE which triggers the capillary action and movement of sample across the membrane. The absorbent pad continuously wicks the sample allowing constant flow of the target analytes during the experiment therefore target molecules interact with the immobilized probe throughout the duration of the test. We let the interaction progress for a certain amount of time, then replaced the sample solution with DI water to rinse the membrane and remove the unbound target.

### Detection and Quantification

We performed quantitative fluorescence experiments using a Nikon AZ100 upright microscope equipped with a 0.5× objective (Plan, NA 0.5, Nikon Corporation, Tokyo, Japan), an epifluorescence filter cube (488 nm excitation, 518 nm emission, Omega Optics, Brattleboro, Vt.), and a 16-bit, cooled CCD camera (Cascade 512B, Photometrics, Tucson, Ariz.) which capture images at the exposure time of 1 s. Images taken from the test zone after each experiment were processed using an in-house MATAB code to quantify the amount of target captured in the test area, m_{T}, effective stacking ratio, p, and ITP residence time, t_{ITP}. We calculated both the mean and standard deviation of the background, determined from a cropped image of the device excluding the test line. The mean-subtracted intensity of each pixel in the test area was then counted as the signal if it was greater than 3 standard deviations of the background, i.e. signal to noise ratio (SNR) of 3. To convert the signal intensity values to the target concentration, we generated a calibration curve by performing three titration experiments. In each titration experiment we measured fluorescence intensity of the membrane surface fully wetted by a known high concentration of the target solution and generated a calibration curve from a linear regression. The calibration experiments were done after performing each set of experiments, on multiple days, and we found the results to be very consistent and repeatable.

### Results and Discussion

### Demonstration of ITP Enhanced LF

After applying the electric field to the system, a highly focused plug of target molecules electromigrates to the test zone where they react with capture molecules immobilized on the surface. ITP increases the target concentration and interaction kinetics of the target-probe which results in generation of a specific fluorescence signal at the test zone. In FIG. 20C, we present five snapshots of a single ITP-LF experiment showing successful capturing of the target using ITP (an example video of this experiment is provided in the supplemental information to Anal. Chem., 87(2), 1009-1017 (2015)). In snapshots a-c, an ITP zone containing highly concentrated target forms and migrates across the membrane. As the ITP zone travels downstream it sharpens and its fluorescence intensity increases demonstrating that the target concentration is increasing. As the plug approaches the test zone, we decrease the applied current and the slow moving plug sweeps over the test zone and binds with the immobilized probes. After the ITP zone passed the test zone (snapshots d-e), we observe a fluorescence signal at the test zone indicating that the target molecules have been captured by the immobilized probes. Any unreacted target molecules electromigrate towards the LE reservoir which effectively serves as a membrane wash step.

### Experimental Determination of the Kinetic and Focusing Parameters

Kinetic parameters, K_{D} and k_{off}, as well as the ITP focusing parameters, p and t_{ITP}, need to be determined in order to compare the experimental results with the model predictions described in equations 1 and 2. To obtain the kinetic parameters we performed a conventional LF assay experiment using 25 mg/l initial target concentration and monitored the signal over time. We measured the captured amount of target in LF assay as a function of time, and fit Equation 2 to these data using the least square method using K_{D} and k_{off} as the fitting parameters. The fitting parameters were determined as, k_{off}=1.75×10⁻³ s⁻¹ and K_{D}=1.42×10⁻⁶M which are consistent with the range of values reported for the interaction of secondary antibodies.

We obtained the ITP focusing parameters, p and t_{ITP}, by conducting independent ITP experiments. We performed ITP experiments and measured the ITP zone velocity, V_{ITP}, width of the ITP plug, δ_{ITP}, and the effective concentration ratio, p. For each ITP experiment we obtained images of the ITP plug prior to reaching the test zone, then converted the intensity values to target concentration using our calibration curve. We fit a Gaussian distribution in the form of A exp(−(x−μ)²/2σ²) to the resulting concentration profile. The resulting fitting parameters are the maximum plug concentration A, mean μ, and the plug width δ_{ITP}=±2σ (two times the plug standard deviation). We experimentally estimated the effective ITP concentration ratio of p=92±12 for I=500 μA using a method described earlier. The ITP velocity was measured by dividing the average displacement distance of the Gaussian fit obtained from twenty pairs of images taken with 1 second image-to-image delays. By dividing the value of V_{ITP} to δ_{ITP} we obtained the characteristic ITP reaction time as t_{ITP}=9.4±0.74 s for I=500 μA, and t_{ITP}=284±47 s for I=50 μA. The plus/minus values represent 95% confidence interval obtained from 3 measurements. We used the later value for our model since the current is set to 50 μA when the ITP zone is crossing the test zone in ITP-LF.

### Capture Enhancement by ITP

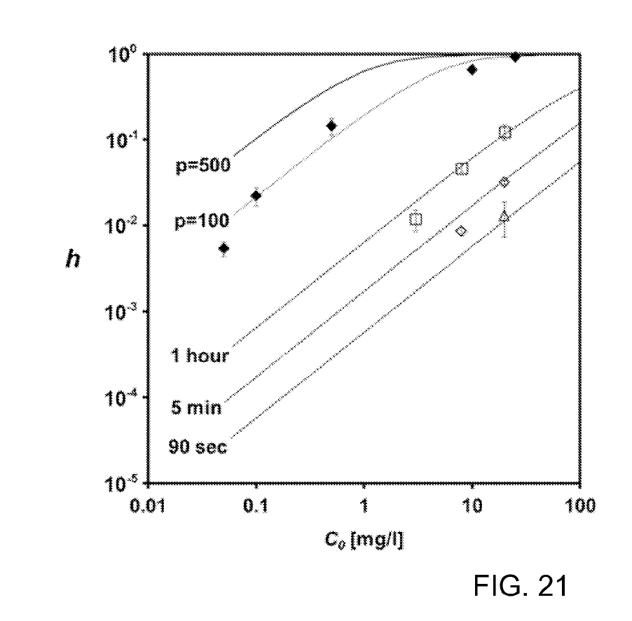

FIG. 21 provides experimental data showing quantitative detection of IgG using ITP-LF (closed diamonds) and its comparison to conventional LF at 90 second (open triangle), 5 minutes (open diamonds), and 1 hour (open squares). Along with experimental data (symbols), we show results of analytical models for the conventional (dashed) LF and for the ITP-LF (solid) assays. ITP creates two orders of magnitude improvement in LOD of LF after 7 minutes assay time. To generate the model curves we set the k_{off}=1.75×10-3 s⁻¹ and K_{D}=1.42×10-6 M, p=92 and t_{ITP}=284 s. The error bars indicate 95% confidence interval for three measurements.

FIG. 21 shows experimental data for the fraction of bound probe h as a function of target concentration for ITP-LF and LF. Assuming one-to-one binding of target and probe, the fraction of bound probe is defined as the surface concentration of the bound probe, C_{C}, normalized by the initial surface concentration of the probe, C_{P0}. We estimated the fraction of bound probe by normalizing the amount of bound target in the test zone with its maximum value. Each ITP-LF takes about 7 minutes to complete with the ITP zone residence time of 284 s. LF data were collected at three different assay times of 90 seconds, 5 minutes and 1 hour. h increases with initial target concentration for both LF and ITP-LF. The amount of captured sample in ITP-LF increases with increasing target concentration, however at concentrations higher than 25 mg/l we did no observe a change in the amount of captured target which suggests that the probe surface was saturated. At each concentration the fraction of bound probe using ITP is higher than the conventional LF since the ITP concentration increases the reaction rate. For example, at 10 mg/l the fraction of bound probe is 6.7×10⁻¹ for ITP-LF and is 8.53×10⁻³ for LF after 5 min. This shows improved capture by ITP concentration by about 80 fold. h_{LF} increases with time and reaches near equilibrium conditions after one hour, which can be seen by taking the derivative of Equation 1 in respect to t.

Along with the experimental data in FIG. 21 we present analytical models (equations 1-2) for ITP-LF and conventional LF. To obtain these theoretical curves, we used the experimentally determined kinetic and focusing parameters as k_{off}=1.75×10⁻³ s⁻¹, K_{D}=1.42 μM, p=92 and t_{ITP}=284 s. For LF, we observe good agreement of predicted trends and our experimental data. For ITP-LF, we notice good agreement at higher concentrations but lower than predicted binding at 0.05 mg/I. We hypothesize that this over prediction of h by the model at low concentration may be due to deviation from the kinetically limited assumption as a result of very low number of moles of target present above the reaction zone. The number of moles of target in the ITP zone here is approximately 8 fmoles. At this condition, roughly one-third of the target molecules have been consumed by the surface reaction, therefore the assumption of constant target concentration may not be valid.

LOD in our system is defined as the lowest target concentration at which the maximum background-subtracted intensity in the test zone is 3 standard deviations away from the background, i.e. SNR=3. In practice, for example, the normalized standard deviation for ITP-LF is ˜8×10⁻⁴ and the fraction of bound target, h, corresponding to LOD is ˜5.4×10⁻³ for ITP-LF and ˜10⁻² for LF. The bound target concentration of ITP-LF at LOD is marginally lower because the electrokinetic “wash” step during ITP effectively removes the unbound target from the surface and improves the SNR. Table 1 lists the LOD extracted from FIG. 21 for ITP-LF and LF at three assay times tested. This table shows that the LOD is improved by a factor of 60, 160, and 400 for 1 hour, 5 minutes, and 90 seconds LF assay times, respectively. The normalized initial target concentration, C_{T0}/K_{D}, at limit of detection is 3.3×10⁻⁴ for ITP-LF which is two orders of magnitude below the lowest value reported for conventional LF assays.

TABLE 1
Detection limit improvement of conventional LF assay using ITP.

| | | | Fold improvement of |
|---|---|---|---|
| Assay time | LOD_{LF} (mg/l) | LOD_{ITP-LF} (mg/l) | detection limit |
| 90 seconds | 20 | 0.05 | 400 |
| 5 minutes | 8 | 0.05 | 160 |
| 1 hours | 3 | 0.05 | 60 |

In addition to improving the detection limits, ITP increases the capturing efficiency of LF assays. In Table 2 we present the capturing ratio of ITP-LF, (N_{C}/N_{T0})_{ITP-LF}, as a function of initial target concentration, C_{T0}, and compare it to the capturing ratio of LF assay, (N_{C}/N_{T0})_{LF}. Capturing ratio is calculated by calculating moles of target captured in the test area, N_{C}, and normalizing it by the initial moles of sample in the sample reservoir, N_{T0}. Capture ratio for LF increases by increasing the initial target concentration and reaches 0.7% at C_{T0}=25 mg/I. Similar trend is observed for ITP-LF with the highest capture ratio of 30% observed at C_{T0}=0.5 mg/I. An apparent decrease in the capturing ratio of ITP-LF is observed at higher concentrations because the test line is saturated due to having excess amount of target molecules in the sample solution, i.e. N_{To} is larger than the initial moles of probe, N_{P0}. Unbound target molecules during ITP=LF travel with the ITP plug toward the LE reservoir as shown in FIG. 20C-d. This suggests that we may be able to increase the capturing efficiency of ITP-LF by using even smaller volumes of the sample. LF assay shows capturing ratios smaller than 1% and we are not able to detect the target at concentrations lower than 8 mg/l which is below the LOD of LF assay. At each concentration, capturing ratio of the conventional ITP-LF assay is one order of magnitude larger than the capturing ratio of LF. This data suggests that integration of ITP with LF may offer the capability in using large sample volumes with very dilute concentration of target.

TABLE 2
Fraction of captured target, N_{C}/N_{T0}, by ITP-LF compared to
that of LF. C_{T0} is the initial target concentration, N_{T0} is the initial
number of moles of target, and N_{P0} is the number of moles
of probe on the surface. The uncertainties represent 95% confidence
interval for three measurements.

| | | | (N_{C}/ | | (N_{C}/ |
|---|---|---|---|---|---|
| C_{T0} ^{a} | N_{P0} ^{b} | (N_{T0})_{ITP-LF} ^{b} | N_{T0})_{ITP-LF} ^{c} | (N_{T0})_{LF} ^{b} | N_{T0})_{LF} ^{c} |
| 0.05 | 0.5 | 0.02 | 11.62 ± 2.29 | 0.06 | No detection |
| 0.1 | 0.5 | 0.04 | 24.05 ± 5.83 | 0.13 | No detection |
| 0.5 | 0.5 | 0.23 | 30.86 ± 6.91 | 0.67 | No detection |
| 10 | 0.5 | 4.6 | 7.01 ± 0.32 | 10.6 | 0.47 ± 0.05 |
| 25 | 0.5 | 11.6 | 3.94 ± 0.08 | 26.6 | 0.70 ± 0.04 |

^{a} in mg/l,
^{b} in fmoles,
^{c} in %

### Colorimetric Detection in ITP-LF

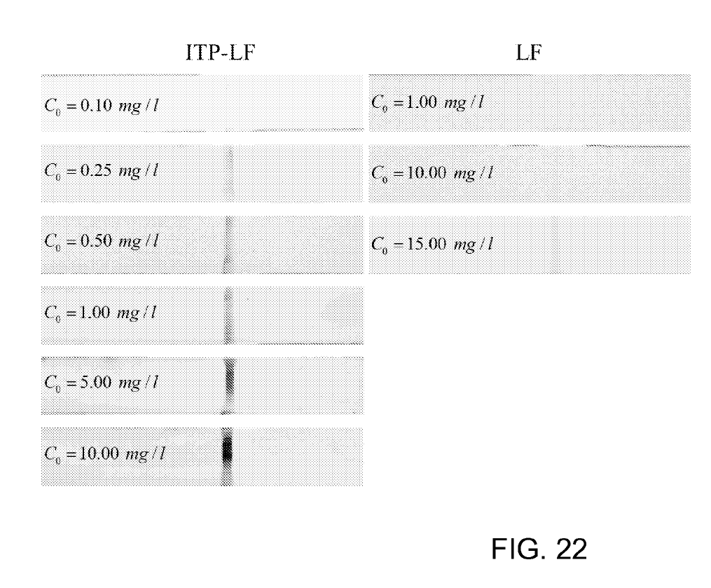

The capability of LF assays to generate an unambiguous and visually read result, i.e. colored band, is critical feature of their success as a POC diagnostic. This is achieved by using detection antibodies labeled with colloidal gold or other colorimetric signal generators such as colored latex particles,⁵³ and colloidal carbon nanoparticles.⁵⁴ In order to show the compatibility of our ITP-LF assay with colorimetric detection, we used IgG labeled with 40 nm colloidal gold (IgG-Au) as our target. We measured effective electrophoretic mobility of IgG-Au at pH 7.4 as 8.9 nm²/Vs. Since this value is larger than the effective mobility of TE ions we used in our quantitative analysis, we kept the same TE composition to perform colorimetric ITP-LF experiments. Generation of a pink-colored band at the test zone indicates successful capturing of the target with the color intensity directly proportional to the amount of sample captured. ITP is able to focus IgG molecules labeled with gold nanoparticles and transport them across the membrane toward the test zone (a video of colorimetric ITP-LF is provided in the SI). FIG. 22 shows snapshots of the paper device used for colorimetric detection of the target using both ITP-LF (left) and conventional LF (right) for the initial target concentration ranging from 0.1-15 mg/I. Here the target is Goat anti Mouse IgG labeled with 40 nm colloidal gold. LF assays are performed for 5 min to be consistent with ITP assay time. The optical density of the test line increases with initial target concentration as expected. Qualitatively, a visually detectable signal was observed using ITP-LF at concentrations as low as 0.1 mg/l while no signal was detected using LF at concentrations lower than 10 mg/l and 10 minutes assay time. Qualitatively, these data show more than 100-fold improvement in detection limit of LF assay using ITP which is consistent with our quantitative fluorescence measurements.

Insights into the Design of an ITP-LF Assay

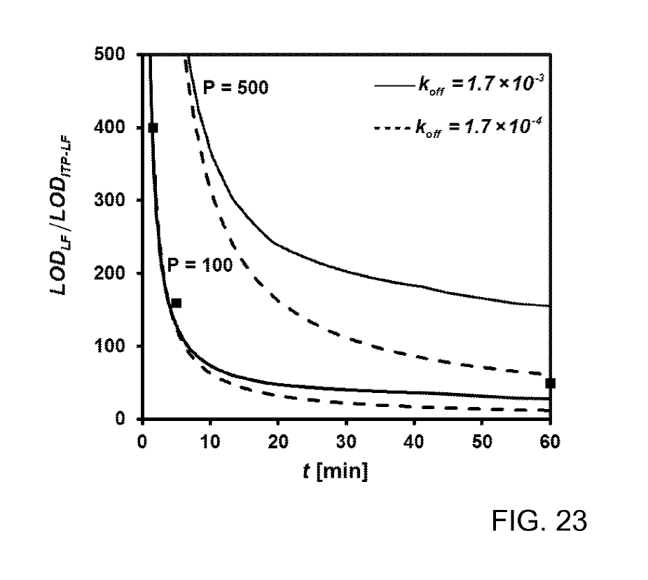

We use our analytical model as a guide for the design of an ITP-enhanced LF assays. FIG. 23 plots the ratio of LOD_{LF}/LOD_{ITP-LF}, predicted by our analytical model, versus the assay time, t. Each value on y-axis shows the level of improvement in LOD of LF. Contours are plotted for t_{ITP}=284 s, p=100 (bottom), p=500 (top), k_{off}=1.75×10⁻³ s⁻¹ (solid lines) and k_{off}=1.75×10⁻⁴ s⁻¹ (dashed lines). We note that contours have no dependence on the K_{D} value showing that ITP improves detection limit of LF by promoting the reaction rate. Solid symbols represent our experimental conditions where p=92, t_{ITP}=284 s and different LF assay time showing good agreement with the model prediction.

In FIG. 23 we present the LOD ratio (LOD_{LF}/LOD_{ITP-LF}) versus the assay time for different effective concentration ratios and off-rate constants. Each value on the y-axis shows the improvement in LOD when using ITP at a given LF assay time. ITP concentration results in a considerable improvement in LOD of the LF assay due to the elevated forward reaction rate as a result of higher target concentration in the test zone. LOD ratio decreases at longer times since the binding of target and probe in kinetically limited LF assay increases with time. ITP improves the LOD of LF assay even at longer times despite the fact the LF reaches equilibrium. For example, at p=100, ITP improves the LOD of LF assay by a factor of 30 at 1 hour assay times and longer. FIG. 23 shows that for shorter assay time much higher LOD ratios can be obtained which means, in addition to the LOD improvement, ITP can speed up the LF reaction. In the Supplemental Information to Anal. Chem., 87(2), 1009-1017 (2015)). we provide a discussion on increasing the reaction rate using ITP. Closed symbols in FIG. 23 represent our experimental conditions where p=92, t_{ITP}=284 s and k_{off}=1.75×10⁻³ s⁻¹, showing good agreement with the model predictions. We hypothesize that the under prediction by the model at 1 hour assay time is due to the fact that the LF may have not reached equilibrium. We believe that at very low target concentrations, LF is limited by the diffusion of the target molecules to the test zone and thus requires longer times, than predicted by the model, to reach equilibrium. FIG. 23 also shows that at higher concentration ratios, e.g. p=500, higher LOD ratios can be achieved, as expected.

We also examined effect of kinetic parameters and observed that the k_{off} has marginal improvement effect on the LOD. For example, at 30 min assay time, increasing the k_{off} by one order of magnitude increases the LOD ratio by less than 2-fold. When the k_{off} is increased the kₒₙ value also increases, at constant K_{D}, therefore the forward reaction rate is increased. We observed that LOD ratio does not depend on the K_{D} value because ITP improves the binding by increasing the forward reaction rate. The equilibrium dissociation constant is a representation of the affinity of two compounds and does not depend on time. For conventional LF assay, the fraction of the bound probe at longer times is directly proportional to the initial target concentration, h_{LF}≈C_{T0}/K_{D} for conditions satisfying C₀*<<1. Similarly, in ITP-LF, a Taylor series expansion for the limiting case of (pC₀*+1)k_{off}t_{ITP}<<1 yields the linear proportionality of h_{ITP}≈pk_{off}t_{ITP} (C_{T0}/K_{D}).³⁷ At the LOD concentration, signal strength, i.e. fraction of the bound probe, is the same for LF and ITP-LF. Therefore, LOD_{LF}z′ h_{LOD}K_{D} and LOD_{ITP-LF}≈k_{LOD}/pkₒₙt_{ITP}. As a result, the LOD ratio scales as pk_{off}t_{ITP} which confirms our model predictions presented in FIG. 23 that the LOD ratio is directly proportional to the ITP parameter pt_{ITP} and the off-rate constant while it is independent of the K_{D} value.

Our scaling analysis and the analytical model show that the amount of captured target for conventional LF assay depends on the normalized initial target concentration, i.e. C_{T0}/K_{D}. On the other hand, for ITP-LF the amount of captured target depends on pk_{off}t_{ITP}(C_{T0}/K_{D}) which includes both antibody-specific parameters, k_{off} and K_{D}, and an ITP condition parameter pt_{ITP}. This offers flexibility in assay design by controlling the concentration ratio and the ITP residence time. For a point of care LF test, known values can be kinetic parameters of the antibody-antigen pair and the desired test time. From this, one can estimate the ITP concentration ratio and residence time required to achieve the desired detection limits. Concentration ratio in ITP can be optimized by carefully selecting the electrolyte system, optimizing the applied electric field and the device length.

### SUMMARY

We demonstrate LOD and assay time improvement of LF assays using ITP. Our method leverages high concentration power of ITP to overcome the slow reaction kinetics of surface binding in LF. We focused target antibodies into a narrow ITP zone (order 100 μm) and transported them across the nitrocellulose membrane to the test zone. Our quantitative analysis show that LOD of LF assays can be improved by 400-fold, 160-fold and 60-fold at assay time of 90 sec, 5 min and 1 hour, respectively. Our qualitative colorimetric measurements show about 100-fold improvement in LOD using ITP, consistent with our quantitative findings. We used an analytical model describing the ITP-enhanced surface reaction in LF assays which confirms our experimental observations and serves as guide toward the design of LF assay enhanced by ITP. By integrating ITP into LF assay we can also capture up to 30% which shows more than one order of magnitude improvement over conventional LF assays. High target extraction and concentration of ITP enables the LF assays to use large volume of sample when the target is very dilute, e.g. blood, and waste water. Moreover, we found that detection limit of LF can be improved by separating and preconcentrating target analytes from 5× diluted mouse serum (data provided in SI) showing the potential of ITP to improve LF with real-world complex biological samples.

1. A device comprising:
a porous matrix having a first end and a second end opposing the first end, the first end and the second end defining a first axis, the porous matrix having a first fluid pathway having a first end and extending to a second end;
a first electrode disposed adjacent the first end of the first pathway; and
a second electrode disposed adjacent the second end of the first pathway.

2. The device according to claim 1, further comprising
a trailing electrolyte, disposed in the porous matrix within the first fluid pathway, the trailing electrolyte comprising an ion and a counterion; and
a leading electrolyte, disposed in the porous matrix within the first fluid pathway, the leading electrolyte comprising an ion and a counterion, the ion of the leading electrolyte having a higher effective electrophoretic mobility than the ion of the trailing electrolyte.

3. The device according to claim 2, wherein
(a) the leading electrolyte is disposed closer to the second end of the first fluid pathway than is the trailing electrolyte;
(b) the trailing electrolyte is disposed within a first fluid disposed within the porous matrix within the first fluid pathway, such that the first electrode is in conductive contact with the first pathway;
(c) the leading electrolyte is disposed within a second fluid disposed within the porous matrix within the first fluid pathway, such that the second electrode is in conductive contact with the first pathway; and/or
(d) the trailing electrolyte is disposed within a first fluid disposed within the porous matrix within the first fluid pathway, and the leading electrolyte is disposed within a second fluid disposed within the porous matrix within the first fluid pathway, the second fluid being disposed closer to the second end of the first fluid pathway than is the first fluid.

4.-8. (canceled)

9. The device of claim 2, further comprising an analyte disposed in the porous matrix, the analyte comprising an analyte ion and an analyte counterion, the ion of the analyte ion having a higher effective electrophoretic mobility than the ion of the trailing electrolyte and a lower effective electrophoretic mobility than the ion of the leading electrolyte.

10. The device of claim 9, wherein the difference between the effective mobilities of the ion of the leading electrolyte and the ion of the trailing electrolyte is at least about 6 nm²V⁻¹s⁻¹.

11. The device of claim 1, further comprising an analyte disposed in the porous matrix, the analyte comprising an analyte ion and an analyte counterion.

12. The device according to claim 10, wherein the analyte is disposed in a first fluid disposed within the porous matrix within the first fluid pathway.

13. The device according to claim 10, wherein the analyte has a concentration at least about four orders of magnitude smaller than the concentration of the leading electrolyte in the second fluid, and a concentration at least about four orders of magnitude smaller than the concentration of the trailing electrolyte in the first fluid.

14. The device of claim 1, wherein the first end of the fluid pathway is disposed at the first end of the porous matrix, and the second end of the fluid pathway is disposed at the second end of the porous matrix.

15. The device according to claim 1, configured for the detection of an analyte, wherein the porous matrix includes a detection zone in the first fluid pathway.

16. The device according to claim 15, wherein the detection zone includes a detection substance capable of interacting with the analyte to provide a detectable change, or wherein the detection zone comprises a series of separated areas of detection sub stance.

17. The device according to claim 15, wherein the detection zone comprises a series of separated areas of detection substance, and wherein the detection substance is the same in each of the separated areas.

18. (canceled)

19. The device according to claim 17, wherein the separated areas of the detection substance have increasing concentrations in the direction from the first end to the second end of the first fluid pathway, or wherein the separated areas of the detection substance have increasing widths in the direction from the first end to the second end of the first fluid pathway.

20.-24. (canceled)

25. The device according to claim 15, further comprising an optical readout system configured to perform an optical measurement on the detection zone.

26. The device according to claim 1, wherein
(a) the porous matrix has an average pore size in the range of about 0.1 μm to about 100 μm;
(b) the porous matrix has a porosity of at least 80%;
(c) the porous matrix has an internal surface area ratio in the range of about 50 to about 200; and/or
(d) wherein the porous matrix is formed from a paper or membrane material.

27.-28. (canceled)

29. (canceled)

30.-34. (canceled)

35. The device of claim 1, wherein
(a) the first fluid pathway further comprises a water-soluble material selected from one of the group comprising polymeric surfactants, charged polymers, poly(vinyl alcohol), poly(alkylene glycol) polymers, Polyethylene glycol (PEG) and Polyvinylpyrrolidone (PVP), Tween-20, Triton-X, polylactams, substituted polyacrylamide derivatives, and water soluble methylhydroxyethyl derivatives of cellulose; and/or
(b) the porous matrix includes a first portion disposed generally along the first fluid pathway, the first portion having a first lateral edge and a second lateral edge opposing the first lateral edge, and at least one tab extending from the first lateral edge or the second lateral edge of the first portion, each of the at least one tab having a second fluid pathway in fluid communication with the first fluid pathway.

36.-39. (canceled)

40. The device of claim 1, wherein
(a) the device includes a substantially water-impermeable cover disposed over the porous matrix;
(b) the porous matrix is disposed in a closeable casing;
(c) the device is configured such that application of electric voltage is initiated by the application of one of the first and second fluids to the device;
(d) configured as a substantially flexible device, the device including a flexible battery on which the porous matrix is disposed;
(e) further comprising an absorbent pad disposed on the porous matrix at the first end of the fluid pathway, the absorbent pad being in fluid communication with the first pathway;
(f) further comprising a reservoir disposed on the top surface of the porous matrix at the first end of the first fluid pathway, the reservoir being in fluid communication with the first pathway;
(g) wherein a first portion of porous matrix has a first zone and a second zone, the first zone being disposed more toward the first end of the porous matrix than the second zone, the first zone having a substantially greater average width than the second zone; and/or
(h) wherein a first portion of porous matrix has a first zone and a second zone, the first zone being disposed more toward the second end of the porous matrix than the second zone, the first zone having a substantially greater average width than the second zone.

41.-48. (canceled)

49. A method of concentrating an analyte in a sample, the method comprising
providing a device according to claim 1, the device including
a trailing electrolyte, disposed in the porous matrix within the first fluid pathway, the trailing electrolyte comprising an ion and a counterion;
a leading electrolyte, disposed in the porous matrix within the first fluid pathway, the leading electrolyte comprising an ion and a counterion, the ion of the leading electrolyte having a higher effective electrophoretic mobility than the ion of the trailing electrolyte; and
an analyte disposed in the porous matrix, the analyte comprising an analyte ion and an analyte counterion, the ion of the analyte ion having a higher effective electrophoretic mobility than the ion of the trailing electrolyte and a lower effective electrophoretic mobility than the ion of the leading electrolyte; and
applying a voltage across the first electrode and the second electrode for a time sufficient to provide an ITP plug.

50.-51. (canceled)

52. A method of concentrating an analyte in a sample comprising:
providing a device comprising
a porous matrix having a first end and a second end opposing the first end, the first end and the second end defining a first axis, the porous matrix having a first fluid pathway having a first end and extending to a second end,
a first electrode, and
a second electrode;
introducing to the first pathway
a first fluid comprising a trailing electrolyte, the trailing electrolyte comprising an ion and a counterion, the first fluid being disposed such that the first electrode is in conductive contact with the first end of the first pathway, and
a second fluid comprising a leading electrolyte, disposed in the porous matrix within the first pathway, the leading electrolyte comprising an ion and a counterion, the ion of the leading electrolyte having a higher effective electrophoretic mobility than the ion of the trailing electrolyte, the second fluid being disposed such that the second electrode is in conductive contact with the second end of the first pathway, and
the analyte, the analyte comprising an analyte ion and an analyte counterion, the ion of the analyte ion having a higher effective electrophoretic mobility than the ion of the trailing electrolyte and a lower effective electrophoretic mobility than the ion of the leading electrolyte; and
applying a voltage across the first electrode and the second electrode for a time sufficient to provide an ITP plug.

53.-60. (canceled)
